(19) United States
(12) Patent Application Publication
(10) Pub. No.: US 2025/0027141 A1
(43) Pub. Date: Jan. 23, 2025

(54) IMPROVED ISOTHERMAL AMPLIFICATION

(71) Applicant: GENETIC SIGNATURES LIMITED, Newton, New South Wales (AU)

(72) Inventor: Douglas MILLAR, Newton, New South Wales (AU)

(21) Appl. No.: 18/713,100

(22) PCT Filed: Nov. 23, 2022
(86) PCT No.: PCT/AU2022/051403

§ 371 Date: May 23, 2024

(30) Foreign Application Priority Data
Nov. 23, 2021 (AU) 2021903771

(51) Int. Cl. C12Q 1/6848; C12Q 1/6853; C12Q 1/689; C12Q 1/6895; C12Q 1/70

(52) U.S. Cl. CPC C12Q 1/6848; C12Q 1/6853; C12Q 1/689; C12Q 1/6895; C12Q 1/70

(57) ABSTRACT

The invention relates to a method of reducing the incidence of false positives, increasing efficiency, and/or increasing the sensitivity of loop-mediated isothermal amplification (LAMP) of a target nucleic acid comprising combining: a plurality of LAMP primers for a target nucleic acid, wherein the plurality of LAMP primers comprises a F3 primer and a B3 primer, wherein both the F3 and B3 primer or the F3 primer comprises a promoter for an RNA polymerase at the 5′ end; dNTPs; a strand-displacing DNA polymerase; a reverse transcriptase; an RNA polymerase; and rNTPs; and optionally a nucleic acid binding dye or probe, to form a LAMP reagent mix, incubating the LAMP reagent mix with the target nucleic acid under conditions suitable for amplification of the target nucleic acid with increased efficiency and/or sensitivity compared to the same amplification performed in the absence of the promoter, RNA polymerase and rNTPs.

### TECHNICAL FIELD

The invention relates to methods and kits for improving isothermal amplification reactions. In particular, the invention relates to methods and kits for increasing the efficiency and/or sensitivity of a loop-mediated isothermal amplification (LAMP) reaction by employing a plurality of LAMP primers, wherein at least one of the LAMP primers comprises a promoter for an RNA polymerase.

### CROSS REFERENCE TO RELATED APPLICATION

This application claims priority to Australian provisional patent application no. 2021903771, filed 23 Nov. 2021, which is incorporated by reference in its entirety.

### BACKGROUND

The detection of a nucleic acid target sequence in a sample usually requires the target sequence to be amplified until the quantity of DNA present is high enough to be measurable. Detection of RNA target sequences, such as the viral RNA, additionally requires transcription of the RNA into DNA before amplification. Polymerase chain reaction (PCR) has played a central role in nucleic acid amplification and nucleic acid detection methods. However, one disadvantage of PCR is the need for a thermal cycler to alter the temperature of a reaction mix in a stepwise fashion every few seconds to minutes to allow DNA denaturing and amplify the DNA fragment and repeat the cycle of denaturing and amplification multiple times.

Isothermal amplification reactions allow detection of a target nucleic acid at a constant temperature and are therefore not constrained by the need for thermal cycling. There are various isothermal amplification methods which all share some common features. For example, because the DNA strand are not heat denatured, isothermal methods rely on a polymerase with strand-displacement activity to enable primer binding and initiation of the amplification reaction. Isothermal amplification methods have been applied to point of care diagnosis of diseases and commercial diagnostic platforms with great success.

Loop-mediated isothermal amplification (LAMP) is an isothermal amplification method designed to detect a target nucleic acid without requiring sophisticated equipment. The LAMP reaction uses a DNA polymerase with strand displacement activity and a set of four to six different primers specifically designed to recognise distinct regions of a DNA or RNA target nucleic acid. The reaction can be performed with limited resources, for example using a water bath for incubation (typically at 60-70° C.), and positive results can be identified visually by turbidity, colorimetric changes, lateral flow readouts, addition of fluorescent DNA-binding dyes or labelled probes. The COVID-19 pandemic has seen LAMP reactions being adopted to rapidly detect SARS-CoV-2 RNA in clinical and residential settings.

Although many isothermal methods and kits now exist, there remains a need for improvement of existing methods and kits with improved efficiency and/or sensitivity.

The present inventors have developed methods and kits for improving the efficiency and/or sensitivity of existing isothermal amplification methods. In particular, the inventors have unexpectedly found that the efficiency and/or sensitivity of LAMP can be improved by adding a LAMP primer comprising an RNA polymerase promoter, and by adding rNTPs along with an RNA polymerase as compared to the same reaction (under the same conditions) in the absence of the LAMP primer comprising the RNA polymerase promoter, rNTPs, and RNA polymerase.

Any discussion of documents, acts, materials, devices, articles or the like which has been included in the present specification is solely for the purpose of providing a context for the present technology. It is not to be taken as an admission that any or all of these matters form part of the prior art base or were common general knowledge in the field relevant to the present technology as it existed before the priority date of each claim of this specification.

### SUMMARY

In a first aspect, there is provided a method of reducing the incidence of false positives, increasing efficiency, and/or increasing the sensitivity of loop-mediated isothermal amplification (LAMP) of a target nucleic acid comprising:
a) combining:
   i) a plurality of LAMP primers for a target nucleic acid, wherein the plurality of LAMP primers comprises a F3 primer and a B3 primer, wherein both the F3 and B3 primer or the F3 primer comprises a promoter for an RNA polymerase at the 5′ end;
   ii) dNTPs;
   iii) a strand-displacing DNA polymerase;
   iv) a reverse transcriptase;
   v) an RNA polymerase; and
   vi) rNTPs; and
   vii) optionally a nucleic acid binding dye or probe,
to form a LAMP reagent mix,
b) incubating the LAMP reagent mix with the target nucleic acid under conditions suitable for amplification of the target nucleic acid with increased efficiency and/or sensitivity compared to the same amplification performed in the absence of the promoter, RNA polymerase and rNTPs.

In one embodiment the probe is specific for a target, for example an Easybeacon™ probe (manufactured by Pentabase).

In one embodiment, the promoter for an RNA polymerase is at the 5′ end of a LAMP primer.

The conditions suitable for amplification of the target nucleic acid may comprise incubating the LAMP reagent mix with the target nucleic acid at a temperature of about 60° C. to about 70° C. for about 10 minutes to about 40 minutes.

The conditions suitable for amplification of the target nucleic acid may comprise incubating the LAMP reagent mix with the target nucleic acid at a temperature of about 65° C. for about 10 minutes to about 40 minutes.

In one embodiment, the conditions suitable for amplification of the target nucleic acid may comprise incubating the LAMP reagent mix with the target nucleic acid at a temperature of about 42° C. for about 1 minute to about 10 minutes, followed by further incubating the LAMP reagent mix with the target nucleic acid at a temperature of about 60° C. to about 70° C. for about 10 minutes to about 40 minutes.

In one embodiment, the conditions suitable for amplification of the target nucleic acid may comprise incubating the LAMP reagent mix with the target nucleic acid at a temperature of about 42° C. for about 1 minute to about 10 minutes, followed by further incubating the LAMP reagent mix with the target nucleic acid at a temperature of about 65° C. for about 10 minutes to about 40 minutes.

In some embodiments, the sequence of the promoter is selected from a T7 promoter of SEQ ID NO. 1, a T3 promoter of SEQ ID NO. 2-4, and a SP6 promoter of SEQ ID NO. 5-6. In some embodiments, random nucleotides can be added at the 5′ or 3′ ends of the promoter sequence to improve amplification.

In some embodiments, the promoter is selected from a sequence having at least 90% sequence identity with the sequence of SEQ ID NO. 1, a sequence having at least 90% sequence identity with the sequence of SEQ ID NO. 2, a sequence having at least 90% sequence identity with the sequence of SEQ ID NO. 3, a sequence having at least 90% sequence identity with the sequence of SEQ ID NO. 4, a sequence having at least 90% sequence identity with the sequence of SEQ ID NO. 5, and a sequence having at least 90% sequence identity with the sequence of SEQ ID NO. 6.

In some embodiments, the RNA polymerase is selected from a T7 RNA polymerase, a T3 RNA polymerase, a SP6 RNA polymerase and an E. coli RNA polymerase.

In some embodiments, the concentration of the rNTPs in the LAMP reagent mix is about 0.25 mM, preferably less than 2.5 mM, and the concentration of the RNA polymerase in the LAMP reagent mix is about 7.5 units per reaction, preferably less than 50 units per reaction.

In one embodiment, the LAMP reagent mix comprises guanidinium hydrochloride (GuHCl). The concentration of GuHCl in the LAMP reagent mix is at least about 30 mM to about 60 mM.

In some embodiments, the target nucleic acid is bisulphite treated.

In some embodiments, the target nucleic acid is in its wild type (WT) form.

In one embodiment, the plurality of LAMP primers comprises a FIP primer, a BIP primer, a LF primer and a LB primer.

In some embodiments the FIP primer, the BIP primer, the LF primer and the LB primer do not comprise a promoter for an RNA polymerase.

In some embodiments, both the F3 primer and the B3 primer comprise a promoter sequence at the 5′ end, and the promoter sequence is selected from a T7 promoter of SEQ ID NO. 1, a T3 promoter of SEQ ID NO. 2-4, and a SP6 promoter of SEQ ID NO. 5-6.

In some embodiments, the F3 primer comprises a promoter sequence at the 5′ end, and the promoter sequence is selected from a T7 promoter of SEQ ID NO. 1, a T3 promoter of SEQ ID NO. 2-4, and a SP6 promoter of SEQ ID NO. 5-6.

In some embodiments, the F3 primer or the B3 primer contains a promoter sequence at the 5′ end, and the promoter sequence is selected from a sequence having at least 90% sequence identity with the sequence of SEQ ID NO. 1, a sequence having at least 90% sequence identity with the sequence of SEQ ID NO. 2, a sequence having at least 90% sequence identity with the sequence of SEQ ID NO. 3, a sequence having at least 90% sequence identity with the sequence of SEQ ID NO. 4, a sequence having at least 90% sequence identity with the sequence of SEQ ID NO. 5, and a sequence having at least 90% sequence identity with the sequence of SEQ ID NO. 6.

The target nucleic acid may be an RNA or DNA. In some embodiments, the RNA or DNA is specific to a pathogen.

The pathogen is selected from a bacterium, virus, fungus and parasite.

The bacterium may be selected from Salmonella spp, Bordetella spp, Campylobacter spp, Clostridium spp, Chlamydia spp, Chlamydophila spp, Listeria spp, Lymphogranuloma spp, Shigella spp, Neisseria spp, Staphylococcus spp, Streptococcus spp, Listeria spp, Leishmania spp, Bacillus spp, Borrelia spp, Rickettsia spp, Corynebacterium spp, Gardnerella spp, Haemophilus spp, Escherichia spp, Helicobacter spp, Klebsiella spp, Legionella spp, Mycobacterium spp, Mycoplasma spp; Moraxella spp, Pasteurella spp, Pneumocystis spp, Pseudomonas spp, Treponema spp, Ureaplasma spp, Vibrio sp, Aeromonas spp, and Yersinia spp.

The virus may be selected from a paraechovirus, rabies virus, measles virus, mumps virus, rubella virus, togaviridae, polyomavirus, papillomavirus, hepadnavirus, poxvirus, adenovirus, picornavirus, hepevirus, calicivirus, reovirus, retrovirus orthomyxovirus, paramyxovirus, coronavirus, Ebolavirus, Filoviridae, Flaviviridae, Rhabdoviridae, Bunyavirales, Arenaviridae, and Hantaviridae. and deltavirus. In one embodiment the coronavirus, is Coronavirus HKU-1, Coronavirus OC43, Coronavirus NL63/229E, or SARS-CoV-2.

The parasite may be selected from Blastocystis spp, Giardia spp, Cryptosporidium spp, Cyclospora spp, Blastocystis spp, Dientamoeba spp, Entamoeba spp, Cryptococcus spp, Enterocytozoon spp, Encephalitozoon spp, Babesia spp, Leishmania spp, Schistosoma spp, Trypanosoma spp, Trichomonas spp, Treponema spp, and Plasmodium spp.

The fungus may be selected from Aspergillus spp, Candida spp, Histoplasma spp, Fusarium spp, Pneumocystis spp, Paracoccoides spp, Coccidioides spp, and Scedosporium spp.

In some embodiments, the sensitivity of the LAMP reaction is increased by at least about 10% to about 30%. In one embodiment, the sensitivity of the LAMP reaction is improved down to a single copy detection level in less than about 18 minutes.

In some embodiments, the efficiency of the LAMP reaction is increased by at least about 5% to about 20%.

In some embodiments, the LAMP is a multiplex reaction wherein the reaction comprises in step a) combining a second plurality of LAMP primers for a second target nucleic acid wherein the second plurality of LAMP primers comprises a F3 primer comprising a promoter for an RNA polymerase at the 5′ end.

In one embodiment, a plurality of LAMP primers for a target nucleic acid is provided, wherein the LAMP primers comprise a F3 primer and a B3 primer each comprising a promoter for an RNA polymerase at the 5′ end, or a F3 primer comprising a promoter for an RNA polymerase and at least one sequence complementary to the target nucleic acid.

In a second aspect, the invention relates to a kit for detecting a loop-mediated isothermal amplification (LAMP) reaction with a target nucleic acid, the kit comprising
i) a plurality of LAMP primers for the target nucleic acid, wherein the plurality of LAMP primers comprises a F3 primer and a B3 primer, wherein both the F3 and B3 primer or the F3 primer comprises a promoter for an RNA polymerase at the 5′ end;
ii) dNTPs;
iii) a strand-displacing DNA polymerase;
iv) a reverse transcriptase;
v) an RNA polymerase;
vi) rNTPs; and
vii) optionally a nucleic acid binding dye or probe.

LAMP reaction products may be detected using a variety of methods known to those skilled in the art, including visual examination or turbidity monitoring of precipitated magnesium pyrophosphate, fluorescence detection of double-stranded DNA (dsDNA) with an intercalating fluorophore or fluorescent probes, bioluminescence reporting through pyrophosphate conversion and by the use of lateral flow devices.

In one embodiment, the kit of the second aspect detects an RNA or DNA specific pathogen.

The plurality of LAMP primers may further comprise a FIP primer, a BIP primer, a LF primer and a LB primer.

In some embodiments the FIP primer, the BIP primer, the LF primer and the LB primer do not comprise a promoter for an RNA polymerase.

In one embodiment, the kit of the second aspect is for detecting a multiplex LAMP reaction.

In some embodiments, the kit of the second aspect for detecting a multiplex LAMP reaction further comprises a second plurality of LAMP primers for a second target nucleic acid, wherein the second plurality of LAMP primers comprises a F3 primer comprising a promoter for an RNA polymerase at the 5′ end.

### Definitions

Throughout this specification, unless the context clearly requires otherwise, the word ‘comprise’, or variations such as ‘comprises’ or ‘comprising’, will be understood to imply the inclusion of a stated element, integer or step, or group of elements, integers or steps, but not the exclusion of any other element, integer or step, or group of elements, integers or steps.

Unless the context requires otherwise or specifically stated to the contrary, integers, steps, or elements of the invention recited herein as singular integers, steps or elements clearly encompass both singular and plural forms of the recited integers, steps or elements.

In the context of the present specification the terms ‘a’, ‘an’ and ‘the’ are used to refer to one or more than one (i.e., at least one) of the grammatical object of the article. By way of example, reference to ‘a sample’ means one sample, or more than one sample.

‘At least one’, as used herein, relates to one or more, in particular 1, 2, 3, 4, 5, 6, 7, 8, 9, 10 or more.

In the context of the present specification the term ‘about’ means that reference to a figure or value is not to be taken as an absolute figure or value, but includes margins of variation above or below the figure or value in line with what a skilled person would understand according to the art, including within typical margins of error or instrument limitation. In other words, use of the term ‘about’ is understood to refer to a range or approximation that a person or skilled in the art would consider to be equivalent to a recited value in the context of achieving the same function or result.

The phrase ‘and/or’ as used herein should be understood to mean ‘either or both’ of the elements so conjoined, i.e., elements that are conjunctively present in some cases and disjunctively present in other cases. As a non-limiting example, a reference to ‘efficiency and/or sensitivity’ can refer, in one embodiment, to efficiency only and in another embodiment, to sensitivity only and in yet another embodiment, to both efficiency and sensitivity.

The term ‘isothermal amplification’ as used herein refers to a process of repetitively copying a target nucleic acid without using heat to separate the strands of any duplex formed during the process. In some embodiments, ‘isothermal amplification’ refers to an amplification reaction performed at a single temperature. In some embodiments, ‘isothermal amplification’ refers to an amplification reaction performed at two different temperatures. Isothermal amplification include, but is not limited to, Loop-mediated isothermal amplification (LAMP), Strand Displacement Amplification (SDA), Helicase Dependent Amplification (HDA), Recombinase Polymerase Amplification (RPA), Whole Genome Amplification (WGA), Rolling Circle Amplification (RCA) and Multiple Displacement Amplification (MDA). It is envisaged that the methods and kits as herein described may be applied in various isothermal amplification techniques such as LAMP, SDA, HDA, RPA, WGA, RCA and MDA.

‘Target nucleic acid’ refers to a nucleic acid sequence or subsequence of a larger nucleic acid (template) that is the object of repetitive copying. The target nucleic acid may be an RNA or DNA, and may be obtained from a biological sample in-vivo or in-vitro. The term ‘target nucleic acid’ as used herein also encompasses mRNA and genomic DNA.

As used herein, the term ‘sample’ is used in its broadest sense. In one embodiment, it is meant to include a representative portion or culture obtained from any source, including biological and environmental sources. Biological samples may be obtained from animals (including humans) and encompass sputum, swab samples, bronchial lavage (BAL), bodily fluids (e.g. blood, blood plasma, serum, CSF, stool or urine), an organ, a tissue, a cell, a sectional portion of an organ or tissue, or a cell isolated from a biological subject (e.g., a region containing diseased cells). Environmental samples include environmental material such as surface matter, soil, mud, sludge, sewage, biofilms, water, and industrial samples. Such examples are not however to be construed as limiting the sample types applicable to the present invention.

The target DNA or RNA may be of eukaryotic origin, prokaryotic origin, viral origin or bacteriophage origin. For example, the target DNA or RNA may be obtained from an insect, a protozoa, a bird, a fish, a reptile, a mammal (e.g., rat, mouse, cow, dog, guinea pig, or rabbit), or a primate (e.g., chimpanzee or human). The target DNA may be a complementary DNA (cDNA) that is generated from an RNA template (e.g., mRNA, ribosomal RNA, siRNA and other variants) using a reverse transcriptase enzyme.

The term ‘primer’ or ‘primer sequence’ refers to an oligonucleotide that hybridizes to a target nucleic acid template to generate a target nucleic acid: primer hybrid and to start nucleic acid synthesis. A skilled person in the art can design and identify suitable primers for any isothermal amplification reaction. Tools for designing primers are known in the art. The primer may be an RNA oligonucleotide, a DNA oligonucleotide, or a chimeric sequence. In one embodiment, a plurality of LAMP primers for detecting LAMP of a target nucleic acid sequence is provided. The term ‘LAMP primers’ as used herein refers to a plurality of primers comprising a first outer primer F3, a second outer primer B3, a first inner primer FIP (or primers F1c and F2), a second inner primer BIP (or primers B1c and B2), a first loop-primer LF, and a second loop-primer LB. In addition to the standard set of LAMP primers, in one embodiment, at least one of the LAMP primers comprises a promoter sequence for an RNA polymerase. For example, the F3 primer or the B3 primer are designed to have an RNA polymerase binding site together with a target recognition sequence. In one embodiment, the RNA polymerase binding site is at the 5′ end of the LAMP primer sequence.

‘RNA polymerase’ as used herein may include, but is not limited to, a T7 RNA polymerase, a T3 RNA polymerase, a SP6 RNA polymerase, and an E. coli RNA polymerase. For example, the term ‘T7 promoter’ relates to a sequence that is recognized by a T7 RNA polymerase, the term ‘T3 promoter’ relates to a sequence that is recognized by a T3 RNA polymerase, and the term ‘SP6 promoter’ relates to a sequence that is recognized by a SP6 RNA polymerase. Once the promoter sequence is recognised by the respective RNA polymerase, the RNA polymerase initiates transcription of RNA from a DNA template. Any suitable RNA polymerase may be included in the methods and kits described herein. In one embodiment, the nucleic acid sequence of the T7 promoter is 20 nucleotides long and set forth in SEQ ID NO: 1. In one embodiment, the nucleic acid sequence of the T3 promoter is either 17, 20 or 24 nucleotides long and set forth in SEQ ID NO:2-4, respectively. In one embodiment, the nucleic acid sequence of the SP6 promoter is 24 or 18 nucleotides long and set forth in SEQ ID NO:5 and 6, respectively.

As used herein, the term ‘strand-displacing DNA polymerase’ refers to a DNA polymerase that has a strand displacement activity apart from its DNA synthesis activity. A strand displacing DNA polymerase can continue DNA synthesis on the basis of the sequence of a nucleic acid template strand by reading the template strand while displacing a complementary strand that is annealed to the template strand. Strand-displacing DNA polymerase used for the isothermal amplification methods may be a proofreading or a non-proofreading DNA polymerase, and it may be thermophilic or mesophilic. Any suitable strand displacing DNA polymerase may be included in the methods and kits described herein. For example, the strand displacing DNA polymerase may be selected from Bst large fragment polymerase, Bst 2.0, Bst 3.0, Bca (exo-), Vent, Vent (exo-), Deep Vent, Deep Vent (exo-), Φ29 phage, MS-2 phage, Z-Taq, KOD, Klenow fragment, GspSSD, GspF, OmniAmp Polymerase, SD Polymerase and any combination thereof.

The terms ‘dNTPs’ and ‘rNTPs’ relate to nucleotides, i.e., deoxyribonucleotide triphosphate and ribonucleotide triphosphate, respectively, as known to the person skilled in the art. The terms also encompass modified forms of dNTPs and rNTPs, provided that these modified forms are recognised by the enzymes having DNA polymerase activity and/or the enzymes having RNA polymerase activity.

As used herein, the term ‘reverse transcriptase’ refers to any DNA polymerase that can copy first-strand complementary DNA (cDNA) from an RNA template. Such enzymes are commonly referred to as RNA-directed or RNA dependent DNA polymerases. In some embodiments, a reverse transcriptase can copy a cDNA strand using either single-stranded RNA or DNA as a template.

Suitable amplification reagents for various isothermal amplification methods are known in the art. For example, the reagent mix for a conventional LAMP reaction comprises a strand-displacing DNA polymerase, LAMP primers, and dNTPs which are added to the target DNA. The reagent mix for a conventional LAMP reaction may further comprise reverse transcriptase if the target nucleic acid is an RNA target. In the context of the present specification, the term ‘LAMP reagent mix’ refers to a reagent mix comprising: i) a plurality of LAMP primers for a target nucleic acid, wherein at least one of the LAMP primers comprises a promoter for an RNA polymerase, ii) dNTPs, iii) a strand-displacing DNA polymerase, iv) a reverse transcriptase, v) an RNA polymerase, and vi) rNTPs.

‘Efficiency’ as used herein is measured based on the time it takes to reach a detectable level of amplification. For example, the higher the amplification efficiency, the less time it takes to reach the amplification result.

‘Sensitivity’ as used herein refers to the limit of detection, i.e., the minimum amount of target nucleic acid that must be present to reliably detect and quantify under a given amplification condition. In one embodiment, sensitivity is expressed as a threshold cycle (Ct) value. The Ct value serves as a tool for calculation of the starting amount of nucleic acid template in a sample and represents the number of cycles at which a fluorescence signal prominently starts to increase from a base line (base signal). In one embodiment, methods and kits of the invention improves sensitivity of isothermal amplification reactions down to a single copy detection level in less than 18 minutes.

In order that the present invention may be more clearly understood, preferred embodiments will be described with reference to the following examples.

### DESCRIPTION OF EMBODIMENTS

### Methods

The inventors found that by adding a promoter for an RNA polymerase to a LAMP primer and adding rNTPs along with an RNA polymerase, the speed and sensitivity of various isothermal amplification methods is improved.

Conventional LAMP is performed at a constant temperature, usually between 60° C. and 70° C. and employs a DNA polymerase with strand displacement activity and a set of four oligonucleotides, termed inner and outer primers, specifically designed to recognize six different recognition sites on the target nucleic acid. The two outer primers play a role in strand displacement during the non-cyclic step only whereas the inner primers include both sense and antisense sequences and contribute to formation of typical LAMP amplification products having stem-loop structures. In addition to the four oligonucleotide primers, the LAMP assay may include two additional primers, the so-called loop primers, to improve amplification efficiency, thereby resulting in a total of six primers per target sequence. Such a combination of different LAMP primers, which span eight distinct sequences on the target nucleic acid, provides better specificity.

The present invention provides a method of increasing the efficiency and/or sensitivity of LAMP by using i) a plurality of LAMP primers for a target nucleic acid, wherein the plurality of LAMP primers comprises a F3 primer and a B3 primer, wherein both the F3 and B3 primer or the F3 primer comprises a promoter for an RNA polymerase at the 5′ end. The methods also comprise ii) dNTPs, iii) a strand-displacing DNA polymerase, iv) a reverse transcriptase, v) an RNA polymerase, vi) rNTPs, and optionally a nucleic acid binding dye or probe. The components are combined to form a LAMP reagent mix. The reagent mix is usually prepared immediately before use. However, it is envisaged that the reagent mix or some components of the reagent mix may be prepared in advance, for example the primers, dNTPs and rNTPs may be premixed and the enzymes added just prior to use.

The plurality of LAMP primers further comprises a FIP primer, a BIP primer, a LF primer and a LB primer.

Preferably only the F3 and B3 primer or the F3 primer comprise a promoter for an RNA polymerase at the 5′ end. That is, no other primer comprises a promoter for an RNA polymerase. For example, the FIP primer, the BIP primer, the LF primer and the LB primer do not comprise a promoter for an RNA polymerase.

The reagent mix will also comprise a buffer solution. Buffer solutions suitable for LAMP reactions are known in the art. One suitable buffer is a pH 8.8 Tris-HCl buffer comprising 20 mM Tris-HCL, 10 mM (NH₄)₂SO₄, 50 mM KCL, 2 mM MgSO₄, 0.1% Tween® 20. A skilled person will be aware of alternative buffers and will understand that the choice of buffer will be partly dependent on the polymerase used. For example the aforementioned buffer is optimised for use with Bst 2.0 DNA polymerase.

Once the LAMP reagent mix is formed, the target nucleic acid is added and the reagent mix and nucleic acid is incubated under conditions suitable for amplification of the target nucleic acid.

In some embodiments the target nucleic acid or sample suspected of containing the target nucleic acid may be bisulphite treated using any method known in the art, thereby facilitating the detection of specific methylation forms of the target nucleic acid.

In embodiments where bisulphite treatment is used the reagent mix and sample may optionally be incubated for a period of 1-15 minutes at about 42° C. before incubating at an elevated temperature of 50-55° C. (for example 53° C.) for 5-40 minutes, for example 15, 20, 30 minutes.

In other embodiments the reagent mix and sample are optionally incubated for a period of 1-15 minutes at about 42° C. before incubating at an elevated temperature of 60-70° C. (for example 65° C.) for 5-40 minutes, for example 15, 20, or 30 minutes.

Preferably, the methods are performed with guanidinium hydrochloride (GuHCl) if required for a particular primer set added to the LAMP reagent mix. GuHCl may be present at a concentration of about 30 nM to about 90 nM, for example about 30 mM, about 40 nM, about 50 nM, about 60 nM, about 70 nM, about 80 nm, or about 90 nM. In some embodiments, the GuHCl is present in the LAMP reagent mix at a concentration of about 60 nM.

The concentrations of the rNTPs and RNA polymerase in the LAMP reagent mix can be optimised. In some embodiments, the concentration of the rNTPs in the LAMP reagent mix is about 0.25 mM, preferably less than about 2.5 mM, and the concentration of the RNA polymerase in the LAMP reagent mix is about 7.5 units per reaction, preferably less than about 50 units per reaction.

As exemplified herein the use of an RNA polymerase promoter sequence as part of a primer sequence improves the efficiency and/or sensitivity of a LAMP reaction. It is envisaged that the same approach can be used to improve the efficiency and/or sensitivity of other isothermal amplification techniques such as Strand Displacement Amplification (SDA), Helicase Dependent Amplification (HDA), Recombinase Polymerase Amplification (RPA), Whole Genome Amplification (WGA), Rolling Circle Amplification (RCA) and Multiple Displacement Amplification (MDA).

### Detection of Isothermal Amplification Products

Progress of the reactions can be measured using any method know in the art to detect nucleic acid. For example, LAMP amplification products can be detected using direct or indirect approaches, such as the nucleic acid binding dye or probe.

Direct detection of LAMP amplification products can employ fluorescence reporting. The approach is based on the use of intercalating dyes, such as ethidium bromide, SYBR Green, EvaGreen and YO-PRO-I. Generally, intercalating dyes are non-sequence-specific fluorescent dyes that exhibit a large increase in fluorescence emission upon binding into double stranded DNA. This property may be used to monitor the nucleic acid amplification in real time by continuously measuring the fluorescence during the LAMP reaction.

More specific detection approaches have been developed and are known in the art (i.e., florescence only in the presence of target nucleic acid sequence, for example, see Real-time Detection and Monitoring of Loop Mediated Amplification (LAMP) Reaction Using Self-quenching and De-quenching Fluorogenic Probes. Gadkar V J, Goldfarb D M, Gantt S, Tilley P A G. Sci Rep. 2018 Apr. 3; 8(1):5548).

Indirect detection of LAMP amplification is well known and rely essentially on the formation of pyrophosphate as a reaction by-product. As LAMP reactions proceed, pyrophosphate ions are released by incorporation of dNTPs into the DNA strand during nucleic acid polymerization. These ions react with divalent metal ions, such as magnesium ions (present in the LAMP reaction mix) to produce a white, insoluble magnesium pyrophosphate precipitate. This product results in a cumulative increase in the turbidity of the reaction solution and pyrophosphate precipitates can be measured in terms of turbidity. Alternatively, the detection of LAMP amplification may be achieved through the incorporation of manganese ions and calcein (also known as fluorescein) in the reaction. Calcein's fluorescence is naturally quenched by binding of manganese ions. Pyrophosphate produced as a by-product of the LAMP reaction removes manganese ions from the buffer through precipitation, and the increased turbidity coupled with restored calcein fluorescence allows easy visual read-out upon excitation with visible or UV light. Another detection format is the enzymatic conversion of pyrophosphate into ATP, which is produced during DNA synthesis, and is monitored through the bioluminescence generated by thermostable firefly luciferase.

In some embodiments, a probe (nucleic acid probe) is used to measure progress of the reaction. Preferably the probe is specific for an amplified target sequence (for example a target sequence from SARS-CoV-2). Nucleic acid probes are often generated by conjugation of one or more fluorophores and quenchers to a nucleic acid that is capable of binding a target sequence. Thus, the type of probe can be used for real-time, sequence-specific quantitation of nucleic acids and comprise a central, target-specific, single-stranded loop flanked by a stretch of five to seven complementary nucleotides that can base-pair to form a stem that terminates in a paired fluorophore and quencher. In the absence of a specific sequence target, fluorescence remains quenched due to the proximity of the fluorophore and quencher at the 5′- and 3′-ends of the probe. Binding of a complementary nucleic acid to the loop leads to a conformational change that forces the fluorophore and stem to unpair and the separation of the fluorophore from the quencher results in sequence-specific fluorescence. Probes produce a signal only when binding to a target, thus providing a direct measure of amplification products.

The methods described herein can be used with any probe known in the art and the skilled person will be able to identify or design suitable target-specific probes for use with the methods.

In one embodiment the methods utilise one or more Easybeacon™ probes.

Multiplexing to amplify several different target nucleic acid sequences simultaneously is known and can be achieved by those skilled in the art.

In one embodiment, the LAMP reaction as described herein allows for multiplex detection of two different target nucleic acid sequences in a single tube/reaction by combining a first and second plurality of LAMP primers and probes targeting first and second target nucleic acids. Preferably, at least one of the first plurality of LAMP primers comprises a promoter for an RNA polymerase at the 5′ end and at least one of the second plurality of LAMP primers comprises a promoter for an RNA polymerase at the 5′ end.

In one embodiment, the LAMP reaction as described herein allows for multiplex detection of three different target nucleic acid sequences in a single tube/reaction by combining a first, second, and third plurality of LAMP primers and probes targeting first, second, and third target nucleic acids. Preferably, at least one of the first plurality of LAMP primers comprises a promoter for an RNA polymerase at the 5′ end, at least one of the second plurality of LAMP primers comprises a promoter for an RNA polymerase at the 5′ end, and at least one of the third plurality of LAMP primers comprises a promoter for an RNA polymerase at the 5′ end.

In one embodiment, the LAMP reaction is amenable to multiplexing with a number of Easybeacon™ probes.

In some embodiments, the LAMP reaction as described herein is performed without extraction of DNA or RNA from the sample and is referred to as an ‘extraction free LAMP’.

### Target Nucleic Acid

The methods described herein can be used with any target nucleic acid such as an RNA or DNA sequence specific to a virus or bacteria or an RNA or DNA sequence specifically associated with a disease or condition.

In some embodiments, the target nucleic acid is from a pathogen or infectious agent, for example where a biological sample contains or is suspected of containing the pathogen. Accordingly, the methods and kits provided herein are useful to detect any known pathogen or infectious agent.

The methods and kits can be used to detect viral, bacterial, fungal or parasite pathogens and infectious agents such as viruses e.g., single stranded RNA viruses, single stranded DNA viruses, Zika virus, HIV, Hepatitis A, B, and C virus, HSV, CMV EBV, HPV, SARS-CoV-2, Influenza A, Influenza B, RSV, Dengue virus 1-4, Chikungunya, Parainfluenza 1-4, adenovirus, human rhinovirus, enterovirus, varicella, e.g. VZV, Herpesvirsus e.g HSV-1, HSV-2, Epstein Barr virus, human metapneumovirus, rotavirus, norovirus groups 1 and 2, Sapovirus, Astrovirus, Bocavirus, Ebolavirus, Filoviridae, Flaviviridae, Rhabdoviridae, Bunyavirales, Arenaviridae, and Hantaviridae.

In one embodiment the viral pathogen is selected from a paraechovirus, rabies virus, measles virus, mumps virus, rubella virus, togaviridae, polyomavirus, papillomavirus, hepadnavirus, poxvirus, adenovirus, picornavirus, hepevirus, calicivirus, reovirus, retrovirus orthomyxovirus, paramyxovirus, coronavirus, Ebolavirus, Filoviridae, Flaviviridae, Rhabdoviridae, Bunyavirales, Arenaviridae, Hantaviridae and deltavirus.

In one embodiment the virus is a Coronavirus, e.g. Coronavirus HKU-1, Coronavirus OC43, Coronavirus NL63/229E, SARS-CoV-2.

In one embodiment the bacteria is selected from Salmonella spp; Bordetella spp., e.g. B pertussis, B. holmesii, B. parapertussis; Campylobacter spp; Clostridium spp., e.g. C. difficile, C. difficile ribotype 027, C difficile ribotype 078; Chlamydia spp., e.g. C. pneumoniae, C. trachomatis; Chlamydophila spp., e.g. C. psittaci; Listeria spp; Lymphogranuloma spp., e.g., Shigella spp; Neisseria spp. e.g. N. gonorrhoea; Staphylococcus spp; Streptococcus spp., e.g. S agalactiae; Listeria spp; Leishmania spp; Bacillus spp; Borrelia spp; Rickettsia spp; Corynebacterium spp; Gardnerella spp; Haemophilus spp., e.g. H. influenzae; Escherichia spp, e.g. E. coli; Helicobacter spp., e.g. H. pylori; Klebsiella spp; Legionella spp., e.g. L pneumophila; Mycobacterium spp., e.g. M. tuberculosis; Mycoplasma spp., e.g. M. pneumonia, M. genitalium, M. homini; Moraxella spp; Pasteurella spp; Pneumocystis spp., e.g. P jirovecii; Pseudomonas spp; Treponema spp; Ureaplasma spp., e.g. U. urealyticum; Vibrio sp., Aeromonas spp., and Yersinia spp., e.g. Y pestis

The parasitic pathogen may be a protozoan or metazoan pathogen such as Plasmodia species, Leishmania species, Schistosoma species, and Trypanosoma species), bacteria (e.g., Mycobacteria, in particular, M. tuberculosis, Salmonella, Streptococci, E. coli and Staphylococci), and fungi (e.g., Candida species and Aspergillus species).

In one embodiment the parasitic pathogen is selected from Blastocystis spp., e.g. B. hominis; Giardia spp., e.g. G. intestinalis, G. lamblia; Cryptosporidium spp, Cyclospora spp., e.g. C. cayetanensis; Blastocystis spp, Dientamoeba spp., e.g. D. fragilis; Entamoeba spp., e.g. E. histolytica; Cryptococcus spp, Enterocytozoon spp., e.g. E. bieneusi; Encephalitozoon spp., e.g. E. intestinalis; Babesia spp; Leishmania spp, Schistosoma spp, Trypanosoma spp.; Trichomonas spp., e.g. T. vaginalis; Treponema spp., e.g. T. pallidum; and Plasmodium spp.

In one embodiment the fungal pathogen is an Aspergillus spp, Candida spp, Histoplasma spp, Fusarium spp, Pneumocystis spp, Paracoccoides spp, Coccidioides spp, or Scedosporium spp.

Methods and kits described herein can be applied for the detection and identification of essentially any nucleic acid-containing organism, or free nucleic acid in the environment. Accordingly, the pathogen or infectious agent can be virtually any pathogen or infectious agent for which genetic information (e.g., gene sequences) is available. In some embodiments, the target nucleic acid is from a human origin. In such cases, the methods and kits can be employed to detect a target nucleic acid in a biological sample such as a biological sample obtained for forensic analysis, for genotyping, and the like.

In some embodiments the disease may include, for example, cancer, diabetes, heart disease, hypertension, neurogenerative and infectious diseases. In some embodiments, the target nucleic acid is associated with a particular genetic condition. For example, the target nucleic acid comprises a single nucleotide polymorphism (SNP) for which PAM identification is advantageous, including, but not limited to, BRCA1/BRCA2 mutations, cystic fibrosis, Duchenne muscular dystrophy and hemochromatosis).

Although conventional LAMP utilises multiple primers for a target nucleic acid a relatively high incidence of false positive results is a known limitation. Conventional LAMP assays may use indirect methods of amplification detection and these, whether real-time or end-point, rely on careful primer design, optimal reaction conditions and robustness testing to negate false positives. In contrast the methods described herein, which use a LAMP primers comprising a promoter for an RNA polymerase, are demonstrated (see Example 9) to reduce the incidence of false positive results when compared to an FDA approved LAMP reaction. Accordingly, the T7 modified LAMP protocols described herein improve the utility of the LAMP method and can reduce the incidence of false positives.

### Kits

The present invention also provides kits for practising the methods disclosed herein. Typically, kits for carrying out the methods of the present invention contain all the necessary reagents to carry out the method. For example, in one embodiment the kit comprises i) a plurality of LAMP primers for a target nucleic acid, wherein the plurality of LAMP primers comprises a F3 primer and a B3 primer, wherein both the F3 and B3 primer or the F3 primer comprises a promoter for an RNA polymerase at the 5′ end, ii) dNTPs, iii) a strand-displacing DNA polymerase, iv) a reverse transcriptase, v) an RNA polymerase, vi) rNTPs, and optionally vii) a nucleic acid binding dye and/or viii) a buffer. The kit can further include instructions for performing a detection and/or identification method provided herein.

In one embodiment a kit may comprise one or more containers containing components for a reaction mix wherein addition of the target nucleic acid to the reaction mix instigates nucleic acid amplification when the reaction mix and target nucleic acid is incubated at an appropriate temperature as descried herein.

Typically, the kits of the present invention will also comprise one or more other containers, containing for example, wash reagents, and/or other reagents as required in the performance of the methods of the invention.

In the context of the present invention, a kit includes any kit in which reagents are contained in separate containers, and may include small glass containers, plastic containers or strips of plastic or paper. Such containers may allow the efficient transfer of reagents from one compartment to another compartment whilst avoiding cross-contamination of the samples and reagents, and the addition of agents or solutions of each container from one compartment to another in a quantitative fashion.

Such kits may also include a container which will accept the test sample, a container which contains the reagents used in the methods, and containers which contain a reagent required for the detection of amplified nucleic acid.

Typically, a kit of the present invention will also include instructions for using the kit components to conduct the appropriate methods. Kits and methods of the invention may be used in conjunction with automated analysis equipment and systems.

For application to detection, identification or quantitation of different target nucleic acids a single kit of the invention may be applicable, alternatively different kits, for example containing reagents specific for each target, may be required. Methods and kits of the present invention find application in any circumstance in which it is desirable to detect, identify or quantitate any target nucleic acid.

The present invention will now be further described in greater detail by reference to the following specific examples, which should not be construed as in any way limiting the scope of the invention.

### EXAMPLES

### Example 1: Materials and Methods

### Primers

A 100 mM stock of each primer (IDT) was prepared in molecular grade water (Sigma RNBJ2199). The primers were then mixed in the ratios shown in Table 1 to give a 10× working solution.

TABLE 1
Primer concentrations

| Primer Name | Concentration | Volume (μL) |
|---|---|---|
| BIP | 100 mM | 16 |
| FIP | 100 mM | 16 |
| B3 | 100 mM | 2 |
| F3 | 100 mM | 2 |
| LB | 100 mM | 4 |
| LF | 100 mM | 4 |
| Water | | 56 |

Tested promoters include the T7 promoter, T3 promoter and SP6 promoter. The nucleic acid sequence of the T7 promoter is 20 nucleotides long and set forth in SEQ ID NO: 1, the nucleic acid sequence of the T3 promoter is either 17, 20 or 24 nucleotides long and set forth in SEQ ID NO:2-4, and the nucleic acid sequence of the SP6 promoter is either 24 or 18 nucleotides long and set forth in SEQ ID NO:5 and 6, as provided in Table 2. The promoter sequences are indicated in bold and underlined in Table 2. Random nucleotides can be added at the 5′ or 3′ ends of the promoter sequences to improve amplification.

TABLE 2
Promoter sequences

| Promoter tail | Promoter Sequence | SEQ ID NO. |
|---|---|---|
| T7 tail | AATTCTAATACGACTCACTATAGGGATAAT | 1 |
| T3-17 tail | AATTCATTAACCCTCACTAAAGATAAT | 2 |
| T3-20 tail | AATTCATTAACCCTCACTAAAGGGAATAAT | 3 |
| T3-24 tail | AATTCGCGCGAAATTAACCCTCACTAAAGATAAT | 4 |
| SP6 tail-1 | AATTCCATACGATTTAGGTGACACTATAGATAAT | 5 |
| SP6 tail-2 | AATTCATTTAGGTGACACTATAGGGG | 6 |

Primers B3 and F3 were synthesised comprising a 5′ tail that contains the binding site of a T7 RNA polymerase, T3 RNA polymerase or SP6 RNA polymerase. These primers were substituted for the conventional LAMP F3 and B3 primers in the following reactions.

LAMP primer sets were designed to the M-gene, N-gene, E-gene and RdRP genes of SARS COV-2, and to the haemagluttinin gene of Influenza A to detect Influenza A H3 targets, as set out in Table 3. Other primers used herein are also set out in Table 3.

TABLE 3
Primer Sequences

| | | SEQ ID |
|---|---|---|
| Primer Name | Primer Sequence | NO. |
| WT-M-F3 | TAGGCTTGATGTGGCTCA | 7 |
| WT-M-R3 | ACAGCTCCGATTACGAGT | 8 |
| WT-M-FIP | CTGGATTGAATGACCACATGGAAGCTACTTCATTGCT | 9 |
| | TCTTTCAG | |
| WT-M-BIP | AACTAACATTCTTCTCAACGTGCCTCACTTTCTAGAA | 10 |
| | GCGGTC | |
| WT-M-LF | GCGTACGCGCAAACAGT | 11 |
| WT-M-LB | ACTCCATGGCACTATTCTGAC | 12 |
| WT-N-F3 | TGGCTACTACCGAAGAGCT | 13 |
| WT-N-R3 | TGCAGCATTGTTAGCAGGAT | 14 |
| WT-N-FIP | TCTGGCCCAGTTCCTAGGTAGTCCAGACGAATTCG | 15 |
| | TGGTGG | |
| WT-N-BIP | AGACGGCATCATATGGGTTGCACGGGTGCCAATGT | 16 |
| | GATCT | |
| WT-N-LF | GAAATACCATCTTGGACTGAGATC | 17 |
| WT-N-LB | ACTGAGGGAGCCTTGAATAC | 18 |
| WT-E-F3 | AATACGACTCACTATAGGGC | 19 |
| WT-E-R3 | TTCAGATTTTTAACACGAGAGT | 20 |
| WT-E-FIP | CGAAAGCAAGAAAAAGAAGTACGCTCCAACTTATG | 21 |
| | TACTCATTCGTTTC | |
| WT-E-BIP | TTGCTAGTTACACTAGCCATCCTTAGGTTTTACAAG | 22 |
| | ACTCACGT | |
| WT-E-LF | ACTATTAACGTACCTGTCTCTTCC | 23 |
| WT-E-LB | ACTGCGCTTCGATTGTGT | 24 |
| B-RdRP-F3 | GTTATATTGTTTTTATAGATTAGTT | 25 |
| B-RdRP-B3 | ACAATTTTATTACCATCAAT | 26 |
| B-RdRP-FIP-3 | ACCTAATTTAACATATAATAAACCAATGAGTGTGTT | 27 |
| | TAAGTATT | |
| B-RdRP BIP | GGAATTTTATTAGGAGATGTTATAATTGTTTATAAA | 28 |
| | TAAAAATACATTAACATTAACC | |
| B-RdRP-LF | CACATAACCATTTCAC | 29 |
| B-RdRP-LB | GTTAATAGTGTTTTTAATATTT | 30 |
| RdRP-F3 | TGAGTGTGTTTAAGTATTG | 31 |
| RdRP-B3 | AAATTACAAACATACTTATCA | 32 |
| RdRP-FIP-IAbRQ | TCTCCTAATAAAATTCCACCTAAGTGAAATGGTT | 33 |
| | ATGTGTG | |
| RdRP-Fd-Cy5 | TCTCCTAATAAAATTCCACCTA | 34 |
| RdRP BIP | AATAGTGTTTTTAATATTTGTTAAGTACAATTTTAT | 35 |
| | TACCATCAAT | |
| RdRP-LF | TTTAACATATAATAAACCA | 36 |
| RdRP LB | GGTTAATGTTAATGTATT | 37 |
| E-F3 | TTTTAATTTTAGTTATGGTAGA | 38 |
| E-B3 | AAACAACAACAAACACAA | 39 |
| E-FIP | CCATATAAAAAATAAAAAACCTATTACTAAATTCCAA | 40 |
| | TGGTATTATTATTGTTGAAG | |
| E-BIP | ATGTTAATAGGAATAGGTTTTTGTAACAAACTAAAAT | 41 |
| | TACTAACC | |
| E-LF | CAAAAAACTTTTTAAACT | 42 |
| E-LB | ATATAATTAAGTTAATTTTTTTTTGG | 43 |
| M-F3 | GGATTATTGGTGGAATT | 44 |
| M-B3 | TTAATATCACAACATCCT | 45 |
| M-FIP | CACACAAACAATCTAAAAAAAACAATAAAAAATGGT | 46 |
| | TTGTTTTGTAGG | |
| M-BIP | GTTTTATGTGGTTATTTAATTTAGTATCCACAAAAAA | 47 |
| | TCACAACTC | |
| M-LF | AACTAAACCACATCAAA | 48 |
| M-LB | TTGATTAGATTGTTTTTAGAA | 49 |
| N-F3 | TGTATAATTTGTTTTTAGTGT | 50 |
| N-B3 | TTTTTATCCTTTTTAAACTCT | 51 |
| N-FIP | AATCAACCACATTCCCAAAAATATTTTAGTGTTTTTT | 52 |
| | GGAATGTTG | |
| N-BIP | TATAGGTGTTATTAAATTGGATGATATTTTATATACA | 53 |
| | TCAATATACTTATTCAACA | |
| N-LF | ACTTCCATACCAATAC | 54 |
| N-LB | GATTTAAATTTTAAAGATTAAG | 55 |
| WT-3-Flu A-F3 | GCASAGAGACTTGAAGATGT | 56 |
| WT-3-Flu A-B3 | CAGTTTAACTGCTYTGTCCA | 57 |
| WT-3-Flu A-FIP | TCAGAGGTGACAGGATTGGTCTAGAATACCGATC | 58 |
| | TTGAGGCT | |
| WT-3-Flu A-BIP | GATTTGTGTTCACGCTCACCGTCCATTCCCATT | 59 |
| | AAGGGCATT | |
| WT-3-Flu A-LF | GTCTTTAGCCATTCCATGAG | 60 |
| WT-3-Flu A-LB | GGACTGCAGCGTAGACG | 61 |
| Color SARS-COV- | AACACAAGCTTTCGGCAG | 62 |
| 2 F3 | | |
| Color SARS-COV- | GAAATTTGGATCTTTGTCATCC | 63 |
| 2 B3 | | |
| Color SARS-COV- | TGCGGCCAATGTTTGTAATCAGCCAAGGAAATTTTGG | 64 |
| 2 FIP | GGAC | |
| Color SARS-COV- | GCATTGGCATGGAAGTCACTTTGATGGCACCTGTGTA | 65 |
| 2 BIP | G | |
| Color SARS-COV- | TTCCTTGTCTGATTAGTTC | 66 |
| 2 LF | | |
| Color SARS-COV- | TTCCTTGTCTGATTAGTTC | 67 |
| 2 LB | | |

### Sample Preparation

Bisulphite (B) and wild-type (WT) reactions were both successful. Bisulphite treatment of samples were performed as follow: Step 1) 150 μL of quantified culture, QAP, RNA or positive clinical sample was added to 250 μL of combined reagent 1+2 from the EasyScreen SP006 sample processing kit (Genetic Signatures); Step 2) samples were heated at 95° C. for 20 minutes, vortexed briefly then spun for 2 seconds and allowed to cool to room temperature; Step 3) the material was then purified on a GS-mini according to the manufacturer's instructions (Genetic Signatures) with a final elution volume of between 60-200 μL; and Step 4) samples were frozen at −70° C. after purification.

Wild-type 4 base purification was performed on a GS-mini according to the manufacturer's instructions (Genetic Signatures) with a final elution volume of between 100-400 μL and samples were frozen at −70° C. after purification.

Reactions were prepared in WarmStart Colorimetric LAMP 2×LAMP Master Mix (New England Biolabs: NEB). This contained 16 mM MgSO₄ and thus 8 mM MgSO₄ in the 1× final reaction. Additional components included SYTO-9 Green Fluorescent Nucleic Acid Stain Mix (Invitrogen), Firescript Reverse Transcriptase (Solis BioDyne), 10× primer mix (IDT), rNTPs (NEB), RNA polymerase (NEB), water and sample. The reagents are set out in Table 4 and the volume of each component is set out in Table 5.

Test LAMP reactions contained RNA polymerase, rNTPs and a primer with RNA polymerase tail (promoter). Control LAMP reactions did not contain RNA polymerase or rNTPs and the primers were included without the RNA polymerase tail. Negative samples ‘no template controls’ (NTCs) contained no templates (no nucleic acid).

Bisulphite treated (Bisulphite LAMP) reactions were optionally first ran at 42° C. for 1-10 minutes in a PCR thermal cycler (BioRad CFX96) and then ran at 53° C. for 30-60 minutes in the same PCR thermal cycler. Alternatively, the samples can be run on a heat block, water bath or incubator. Fluorescence was measured once per minute to assess reaction progress.

Wild type 4 base LAMP reactions were optionally first ran at 42° C. for 1-10 minutes in an PCR thermal cycler (BioRad CFX96) and then ran at 65° C. for 30 minutes in the same PCT thermal cycler. Alternatively, the samples can be run on a heat block, water bath or incubator. Fluorescence was measured once per minute to assess reaction progress.

Target nucleic acids including LAMP primer sets are listed in Table 3.

TABLE 4
Reagents

| Supplier | Reagent |
|---|---|
| NEB | WarmStart ® Colorimetric LAMP 2X Master |
| | Mix (DNA & RNA) Catalog#: M1800L |
| NEB | Bst 2.0 DNA Polymerase Catalog #: M0537S |
| NEB | Bst 3.0 DNA Polymerase Catalog #: M0374S |
| NEB | Bst 2.0 WarmStart ® DNA Polymerase Catalog #: M0538S |
| NEB | T7 RNA Polymerase Catalog #: M0251S |
| NEB | SP6 RNA Polymerase Catalog #: M0207S |
| NEB | T3 RNA Polymerase Catalog #: M0378S |
| NEB | Ribonucleotide Solution Mix Catalog #: N0466S |
| NEB | Deoxynucleotide (dNTP) Solution Mix Catalog #: N0447S |
| Invitrogen | SYTP ™9 Green Fluorescent Nucleic Acid Stain Mix |
| | Catalog #: S34854 |
| Solis | Firescript Reverse Transcriptase Catalog #: 06-13-00050 |
| BioDyne | |

TABLE 5
Components

| Component | Volume (μL) |
|---|---|
| X2 Colorimetric LAMP mix | 7.5 |
| 10X Primer mix | 0.5-1.75 |
| Syto-9 (diluted 1/100) | 0.5 |
| Firescript | 0.15 |
| RNA polymerase | 0.1-0.5 |
| ribonucleotides | 0.075-0.3 |
| Water | To 14 μL |
| Sample | 1-4 μl |

### Example 2: Influenza A LAMP

LAMP reactions were performed on a number of Influenza A positive clinical samples from St Vincent's Hospital Sydney, Australia. Table 6 shows the results of a wild type 4 base LAMP Influenza H3 typing assay performed on these samples.

TABLE 6
Results of a wild type 4 base LAMP assay

| | Time (minutes) | | | |
|---|---|---|---|---|
| Sample # | Control LAMP | Primer F3-T7 | Primer B3-T7 | Primer B3-SP6 |
| 1 | N/A | 28.94 | 24.74 | 24.49 |
| 2 | N/A | 26.17 | 29.59 | 28.94 |
| 3 | 20.46 | 17.86 | 17.87 | 19.53 |
| 4 | N/A | N/A | 27.78 | 27.67 |
| 5 | 29.8 | N/A | N/A | N/A |
| 6 | N/A | N/A | N/A | N/A |
| 7 | N/A | 17.96 | 16.71 | N/A |
| 8 | 23.64 | 16.63 | 17.59 | 16.38 |

LAMP primers F3 and B3 were synthesised with either T7 or SP6 tails (T7 or SP6 promoters) and compared to the control LAMP reaction. As shown in Table 6, the data using the tailed primers resulted in both an increase in the number of positive samples detected and a reduction in the time to result.

### Example 3: SARS-CoV-2 LAMP

LAMP reactions were performed on a 2021 Quality Control for Molecular Diagnostics (QCMD) SARS-CoV-2 panel. Table 7 shows the results of a wild type 4 base LAMP assay performed on these samples.

TABLE 7
Results of a wild type 4 base LAMP assay

| | | SARS-COV-2 N-gene target (minutes) | | | |
|---|---|---|---|---|---|
| QCM | Concentration | | GSL-LAMP | GSL-LAMP | GSL-LAMP |
| CD # | Copies/mL | LAMP | T7 | SP6 | T3 |
| 1 | 12,589 | 22.60 | 19.24 | 16.22 | 17.02 |
| 2 | 316 | 24.33 | 20.17 | 19.12 | N/A |
| 3 | 100 | N/A | N/A | N/A | N/A |
| 4 | 794 | N/A | N/A | 19.34 | 20.85 |
| 5 | 1,259 | N/A | N/A | 20.19 | 20.43 |
| NTC | N/A | N/A | N/A | N/A | N/A |

Wild type 4 base LAMP primers were used to amplify the quantified QCMD panels. F3 primers were synthesised containing T7, SP6 or T3 5′ tails and compared to a conventional LAMP assay. As shown in Table 7, the reactions performed using LAMP with the RNA polymerase tails resulted in a quicker time to result. More importantly an increase in sensitivity was generated with the SP6 assay detecting 4 of the 5 samples while conventional LAMP only detected 2 of the 5 samples.

### Example 4: Cultured SARS-CoV-2 LAMP

LAMP reactions were performed using bisulphite converted cultured SARS-CoV-2 samples from St Vincent's Hospital Sydney, Australia.

TABLE 8
Data for tailed primers

| | N-gene | | | |
|---|---|---|---|---|
| Copies | Control LAMP | T7-LAMP | SP6-LAMP | T3-LAMP |
| 750000.00 | 13.98 | 10.64 | 10.74 | 10.81 |
| 75000.00 | 15.86 | 11.92 | 12.12 | 12.23 |
| 7500.00 | 17.49 | 13.14 | 13.14 | 13.36 |
| 750.00 | 19.24 | 14.49 | 14.54 | 14.86 |
| 75.00 | 20.08 | 15.28 | 15.22 | 15.67 |
| 7.50 | 22.09 | 17.15 | 17.54 | 17.06 |
| 0.75 | N/A | 19.08 | 18.98 | 20.42 |
| 0.08 | N/A | N/A | N/A | 21.66 |

LAMP primers were synthesised containing T7, SP6 or T3 5′ tails and compared to the control (i.e., conventional) LAMP reaction. As shown in Table 8, data using the tailed primers resulted in both an increase in the number of positive samples detected and a reduction in the time to result. Table 8 shows that when using the tailed primers an increase in sensitivity of at least 10-fold can be achieved.

### Example 5: Primer Titration

Optimisation of the T7-LAMP (detection of N-gene) primer concentration reduced the time of the first positive result using 75,000 copies from about 17 minutes down to 10.76 minutes and improved the sensitivity down to single copy detection levels in less than 18 minutes (Table 9). The data show when the optimal primer concentration of 1.75× is used a 10-fold increase in sensitivity can be achieved compared to the control LAMP. Bisulphite and the Wild type T7-LAMP reactions had similar sensitivity and can detect single copy targets when optimised.

TABLE 9
Primer titration

| Copies | 0.5X | 0.75X | 1X | 1.25X | 1.5X | 1.75X | 2X |
|---|---|---|---|---|---|---|---|
| 75000.00 | 17.10 | 13.65 | 12.04 | 11.34 | 11.04 | 10.76 | 11.08 |
| 7500.00 | 19.65 | 15.72 | 13.77 | 12.85 | 12.43 | 12.31 | 12.46 |
| 750.00 | 21.92 | 17.40 | 15.16 | 14.37 | 14.01 | 13.54 | 14.03 |
| 75.00 | 25.05 | 19.19 | 17.01 | 15.95 | 15.39 | 15.05 | 15.56 |
| 7.50 | 28.10 | 22.24 | 19.00 | 18.11 | 17.18 | 17.26 | 19.14 |
| 0.75 | N/A | N/A | N/A | N/A | N/A | 17.65 | N/A |
| 0.08 | N/A | N/A | N/A | N/A | N/A | N/A | N/A |

### Example 6: Guanidinium Hydrochloride

Addition of Guanidinium Hydrochloride (GuHCl) had a favourable effect in increasing the speed and sensitivity of the T7-LAMP reaction.

TABLE 10
Effect of Guanidinium Hydrochloride

| | N-gene | | |
|---|---|---|---|
| Copies | 0 GuHCl | 30 mM GuHCl | 60 mM GuHCl |
| 750000.00 | 12.76 | 11.38 | 10.03 |
| 75000.00 | 14.51 | 13.09 | 11.43 |
| 7500.00 | 15.41 | 13.87 | 12.27 |
| 750.00 | 17.62 | 16.03 | 13.81 |
| 75.00 | N/A | 18.07 | 15.99 |
| 7.50 | N/A | N/A | 17.64 |
| 0.75 | N/A | N/A | N/A |
| 0.08 | N/A | N/A | N/A |

### Example 7: Optimisation of rNTPs and RNA Polymerase Concentration

Optimisation of the T7-LAMP (detection of N-gene) using T7 RNA polymerase (50,000 U/mL) titration showed that an increase in positivity is seen when using 10, 7.5 and 5 units of polymerase in the reaction compared to 12.5 units and no T7 added when compared to the control reaction. However, when 10 units were added to the reaction there is a time lag of over 8 minutes for sample #8.

TABLE 11
Effect of T7 RNA polymerase titration

| | T7 RNA polymerase titration | | | | | |
|---|---|---|---|---|---|---|
| | | 0.25 μl | 0.20 μl | 0.15 μl | 0.1 μl | 0 μl |
| Sample# | Control | T7 | T7 | T7 | T7 | T7 |
| 1 | 21.5 | 21.41 | 22.42 | 21.6 | 22.17 | 22.43 |
| 2 | 24.04 | 24.59 | 26.47 | 29.26 | 23.34 | 25.59 |
| 3 | 19.37 | 20.07 | 19.86 | 19.34 | 19.43 | 19.12 |
| 4 | 29.51 | 24.82 | 26.74 | 25.66 | 26.67 | 25.05 |
| 5 | 18.01 | 18.54 | 18.65 | 18.09 | 18.5 | 18.25 |
| 6 | N/A | N/A | N/A | N/A | N/A | N/A |
| 7 | N/A | N/A | N/A | N/A | N/A | N/A |
| 8 | N/A | N/A | 31.67 | 23.06 | 24.94 | N/A |

Optimisation of the T7-LAMP (detection of N-gene) using rNTP (25 mM) titration showed that the addition of about 0.175 μl of rNTP to the reaction was in the optimal range resulting in the detection of 5 samples compared 0.2 μl that detected 4 samples. When no rNTPs were added to the reaction, the time to result was delayed by up to 15 minutes and only 3 samples were detected as positive. Sample #2 was detected at all concentrations of rNTP except at the highest concentration of rNTP (0.2 μl) and in the sample that had no rNTP added. A delay of up to 12 minutes was shown when 0.1 μl was added.

TABLE 12
Effect of ribonucleotide titration

| | Ribonucleotide titration | | | | | |
|---|---|---|---|---|---|---|
| | 0.2 μl | 0.175 μl | 0.15 μl | 0.125 μl | 0.1 μl | 0 μl |
| Sample# | rNTP | rNTP | rNTP | rNTP | rNTP | rNTP |
| 1 | 29.05 | 30.5 | 30.15 | 30.55 | 33.09 | 45.6 |
| 2 | N/A | 32.35 | 38.11 | 32.82 | 46.61 | N/A |
| 3 | 27.12 | 27.18 | 29.26 | 30.46 | 31.37 | 39.8 |
| 4 | 35.54 | N/A | N/A | N/A | N/A | N/A |
| 5 | 23.68 | 25.01 | 26.6 | 27.85 | 28.96 | 36.35 |
| 6 | N/A | N/A | N/A | N/A | N/A | N/A |
| 7 | N/A | N/A | N/A | N/A | N/A | N/A |
| 8 | N/A | 40 | N/A | N/A | N/A | |

### Example 8: Probe Based LAMP-Multiplexing

Multiplexing of the LAMP reaction was performed and involved attaching a fluorescent dye to the 5′ end of the FIP primer and a second primer complementary to the FIP was synthesised containing a 3′ quencher. When amplification occurs the complementary oligo is displaced and fluorescence is released. See below an example for the N-gene.

N-FIP-Fam
Fam-AACAATAAACCAAAACACAATATTATTAAATAAACGAATGTAGTG
GGGTGTGA
N2-Fd-IBQ
GTTTATTTAATAATATTGTGTTTTGGTTTATTGTT-IBQuencher

Another approach is to design conventional Easybeacon™ probes to the regions in the target nucleic acid that are free from priming sites or using non-essential primers such as the LB and LF primers as probe binding sites. These probes would then bind and subsequently be displaced upon the synthesis of new strands of DNA.

To determine the compatibility of the LAMP assay with multiplexing, Easybeacon™ probes were synthesised to the following SARS-CoV-2 regions: RdRP, Orf1a, As1e, N-gene, E-gene, and M-gene, as well as an endogenous human control (12S rRNA). Easybeacon™ probes contain Intercalating Pseudo Nucleotides (IPNs) that improve the efficiency of binding to the target sequences. The composition of the probe based singleplex reactions are shown in Table 13.

TABLE 13
Composition of the probe based LAMP assay-Singleplex

| | Volume per reaction |
|---|---|
| | Singleplex |
| X2 LAMP color Mix | 7.5 μL |
| Primer mix (1.6 μM FIP/BIP, 0.1 μM F3 + T7, | 0.75 μL |
| 0.1 μM F3, 0.4 μM LF/LB) | |
| 20 μM probe | 0.5 μL |
| FireSCript RT | 0.15 μL |
| rNTPs | 0.2 μL |
| T7 RNA polymerase | 0.2 μL |
| 400 mM Guanidinium Hydrochloride | 1.5 μL |
| Water | 2.2 μL |

Probes were designed to both the LB (loop back) and LF (loop forward) regions of the LAMP reactions. All probes were tested in singleplex in the first instance to determine the levels of fluorescence obtained using probe-based chemistry using SARS-CoV-2 clinical samples. The performance (measured in minutes) of 5 different fluorescent probes using SARS-CoV-2 clinical samples in singleplex are shown in Table 14. The data below indicate that all regions and fluorophores produce strong signals in the LAMP reaction, some of which are stronger than typical RT-PCR signals.

TABLE 14
Performance of individual probes using SARS-COV-2 clinical samples

| | Single-Plex reactions | | | | |
|---|---|---|---|---|---|
| | As1e | JRM-N gene | Gene-N2 | Yu ORF1a | Thol ORF1a |
| Sample # | Fam | TxR | TxR | Cy5 | Cy5 |
| 1 | 13.9 | 15.75 | 19.18 | N/A | 13.37 |
| 2 | 13.6 | 16.45 | 19.19 | N/A | 13.56 |
| 3 | N/A | N/A | N/A | N/A | N/A |
| 4 | 12.4 | 15.41 | 16.77 | 11.61 | 11.58 |
| 5 | 14.49 | 16.65 | 17.23 | 13.23 | 14.51 |
| 6 | 12.46 | 16.18 | 17.17 | 28.32 | 13.03 |
| 7 | 10.58 | 13.3 | 14.6 | 10.08 | 10.58 |
| 8 | 10.59 | 13.32 | 14.45 | 10.14 | 11.15 |

To determine if the LAMP assay could be multiplexed, various combinations of primers and probes were tested in singleplex, duplex and triplex. The probes used in the assay were Easybeacon™ probes (manufactured by Pentabase). The compositions of the LAMP multiplex mixes are listed in Table 15. The data below shows that multiplex reactions performed well in triplex although with a slightly delayed time to result.

TABLE 15
Composition of the probe based LAMP assay-Multiplex

| | Volume per reaction | | |
|---|---|---|---|
| | Singleplex | Duplex | Triplex |
| X2 LAMP color Mix | 7.5 μL | 7.5 μL | 7.5 μL |
| Primer mix-1 (1.6 μM FIP/BIP, | 0.75 μL | 0.75 μL | 0.75 μL |
| 0.1 μM F3 + T7, 0.1 μM F3, | | | |
| 0.4 μM LF/LB) | | | |
| Primer mix-2 (1.6 μM FIP/BIP, | | 0.75 μL | 0.75 μL |
| 0.1 μM F3 + T7, 0.1 μM F3, | | | |
| 0.4 μM LF/LB) | | | |
| Primer mix-3 (1.6 μM FIP/BIP, | | | 0.75 μL |
| 0.1 μM F3 + T7, 0.1 μM F3, | | | |
| 0.4 μM LF/LB) | | | |
| Primer mix-4 (1.6 μM FIP/BIP, | | | |
| 0.1 μM F3 + T7, 0.1 μM F3, | | | |
| 0.4 μM LF/LB) | | | |
| 20 μM probe 1 | 0.5 μL | 0.5 μL | 0.5 μL |
| 20 μM probe 2 | | 0.5 μL | 0.5 μL |
| 20 μM probe 3 | | | 0.5 μL |
| 20 μM probe 4 | | | |
| FireSCript RT | 0.15 μL | 0.15 μL | 0.15 μL |
| rNTPs | 0.2 μL | 0.2 μL | 0.2 μL |
| T7 RNA polymerase | 0.2 μL | 0.2 μL | 0.2 μL |
| 400 mM Guanidinium Hydrochloride | 1.5 μL | 1.5 μL | 1.5 μL |
| Water | 2.2 μL | 0.95 μL | 0 μL |

TABLE 16
Performance of individual probes using SARS-CoV-2 clinical samples in duplex

| | Duplex reactions | | | | | | | | | |
|---|---|---|---|---|---|---|---|---|---|---|
| | As1e + | | As1e + | | As1e + | | As1e + | | JRM-N gene + | |
| | JRM-N-gene | | Gene N2 | | Yu ORF1a | | Thol ORF1a | | Yu ORF1a | |
| Sample # | Fam | TxR | Fam | TxR | Fam | Cy5 | Fam | Cy5 | TxR | Cy5 |
| 1 | 15.64 | 17.9 | N/A | 29.25 | 16.88 | N/A | 18.06 | N/A | 19.08 | N/A |
| 2 | 15.04 | 17.04 | 18.03 | 20.03 | 14.43 | 16.31 | 15.11 | 17.78 | 16.45 | 14.06 |
| 3 | 22 | 24.62 | N/A | 23.38 | N/A | N/A | N/A | N/A | 20.65 | 17.14 |
| 4 | 13.75 | 15.62 | 15.22 | 17.02 | 13.19 | 14.58 | 13.3 | 19.38 | 17.42 | 13.45 |
| 5 | 14.31 | 16.62 | 15.56 | 17.45 | 16.46 | N/A | 22.86 | 19.84 | 18 | N/A |
| 6 | 16.29 | 18.69 | 17.7 | 19.57 | 14.76 | 15.76 | 14.12 | 15.9 | 16.48 | 13.56 |
| 7 | 12.56 | 14.19 | 13.57 | 14.76 | 11.44 | 13.2 | 11.67 | 13.3 | 13.02 | 11.96 |
| 8 | 12.44 | 13.59 | 14.22 | 15.69 | 11.61 | 13.29 | 12.01 | 14.19 | 13.9 | 12.16 |

TABLE 17
Performance of individual probes using
SARS-CoV-2 clinical samples in triplex

| Triplex reactions | |
|---|---|
| As1e + | As1e + |
| JRM-N gene + | Gene-N2 + |
| Yu ORF1a | Yu ORF1a |

| Sample # | Fam | TxR | Cy5 | Fam | TxR | Cy5 |
|---|---|---|---|---|---|---|
| 1 | 16.85 | 19.61 | N/A | 18.06 | 21.37 | N/A |
| 2 | 18.05 | 20.25 | 18.3 | 17.75 | 18.97 | 17.67 |
| 3 | 17.63 | 20.24 | N/A | N/A | 22.05 | N/A |
| 4 | 15.35 | 17.53 | 21.15 | 16.58 | 18.2 | 18.05 |
| 5 | 15.88 | 18.19 | N/A | 18.83 | 20.45 | 18.64 |
| 6 | 15.37 | 17.27 | 16.56 | 18.12 | 19.23 | 18.15 |
| 7 | 13.06 | 14.55 | 15.33 | 14.3 | 15.8 | 15.89 |
| 8 | 13.65 | 14.88 | 14.89 | 14.91 | 16.07 | 15.82 |

The LAMP assay was also tested by incorporation of an endogenous human control, the 12S human rRNA target (Table 18).

TABLE 18
Performance of fourplex detection incorporating
the endogenous human control

| | Fluoro | Ct |
|---|---|---|
| E-gene | Cy5 | 12.88 |
| RdRP | Fam | 13.6 |
| 12SrRNA | Hex | 13.29 |
| N-gene | Texas Red | 11.84 |

Table 18 shows that it is possible to multiplex three individual targets (the SARS-CoV-2 E-gene, RdRP and N-genes) and an internal process control (human 12S rRNA) to produce a complete assay.

### Comparison of Multiplexed GSL LAMP and Real-Time PCR

### Prospective Study

In the Prospective Study, 121 SARS-CoV-2 positive samples were freshly collected from a local hospital diluted in UTM and split in two. One set was purified using the TGA approved GSL sample preparation method (SP012) and then amplified by RT-PCR using the TGA approved RP012 kit. The second set was purified using the GS-mini according to the manufacturer's instructions, and amplified with a QuadPlex GSL Real Time LAMP assay containing the As1e, RdRP, N and M-genes as targets (see Tables 20 and 21). Presumptive positives are samples that were positive for only 1 of 2 genes in RT-PCR or 1 of 3 genes in the LAMP assay.

TABLE 19
Composition of the Multiplex reactions

| | Volume |
|---|---|
| | per reaction |
| X2 LAMP color Mix | 7.5 μL |
| As1e Primer mix (1.6 μM FIP/BIP, 0.1 μM F3 + T7, | 0.75 μL |
| 0.1 μM F3, 0.4 μM LF/LB) | |
| RdRP Primer mix (1.6 μM FIP/BIP, 0.1 μM F3 + T7, | 0.75 μL |
| 0.1 μM F3, 0.4 μM LF/LB) | |
| N-Gene Primer mix (1.6 μM FIP/BIP, 0.1 μM F3 + T7, | 0.75 μL |
| 0.1 μM F3, 0.4 μM LF/LB) | |
| M-gene Primer mix (1.6 μM FIP/BIP, 0.1 μM F3 + T7, | 0.75 μL |
| 0.1 μM F3, 0.4 μM LF/LB) | |
| 20 μM probe As1e LF-probe (Fam) | 0.1 μL |
| 20 μM probe As1e LB-probe (Fam) | 0.1 μL |
| 20 μM probe RdRP LF-probe (Fam) | 0.1 μL |
| 20 μM probe RdRP LB-probe (Fam) | 0.1 μL |
| 20 μM probe N-gene LF-probe (TxR) | 0.1 μL |
| 20 μM probe N-gene LB-probe (TxR) | 0.1 μL |
| 20 μM probe M-gene LF-probe (Cy5) | 0.1 μL |
| 20 μM probe M-gene LB-probe (Cy5) | 0.1 μL |
| FireScript RT | 0.15 μL |
| rNTPs | 0.2 μL |
| T7 RNA polymerase | 0.2 μL |
| 400 mM Guanidinium Hydrochloride | 1.5 μL |

TABLE 20
Typical results of the RT-PCR vs LAMP multiplex comparison

| | | | | GSL LAMP Time minutes | | |
|---|---|---|---|---|---|---|
| | SP012 RT-PCR Ct Value | | | As1e/ | | |
| Sample# | N-Gene | M-gene | 12S rRNA | RdRP | N-gene | M-gene |
| 21 | 33.55 | 33.58 | 22.79 | 12.13 | 12.69 | 11.96 |
| 22 | 37.23 | 38.49 | 22.14 | 13.23 | 14.8 | 13.65 |
| 23 | 36.13 | 37.05 | 22.21 | 13.04 | 14.31 | 13.5 |
| 24 | 31.43 | 31.85 | 23.73 | 11.32 | 12.05 | 11.17 |
| 25 | 39.89 | 41.11 | 26.21 | 13.95 | 14.56 | N/A |
| 26 | 34.42 | 34.18 | 22.27 | 13.07 | 14.8 | 12.71 |
| 27 | 35.38 | 35.29 | 24.47 | 12.7 | 13.25 | 12.07 |
| 28 | 32.53 | 32.24 | 33.44 | 11.89 | 12.3 | 11.24 |
| 29 | 28.89 | 29.23 | 33.72 | 10.55 | 11.15 | 10.27 |
| 30 | 38.34 | 45.89 | 18.73 | 14.51 | 16.22 | N/A |
| 31 | 32.16 | 32.64 | 19.01 | 11.69 | 12.34 | 11.88 |
| 32 | 37.31 | 37.88 | 29.4 | 21.12 | 16.01 | 20.96 |
| 33 | 38.43 | 40.03 | 23.7 | 12.97 | 16.92 | 12.46 |
| 34 | 34.17 | 34.56 | 27.63 | 12.06 | 12.42 | 11.55 |
| 35 | 33.33 | 33.49 | 22.31 | 11.43 | 12.14 | 11.25 |
| 36 | 27.34 | 27.68 | 21.52 | 10.07 | 10.43 | 10.05 |
| 37 | 43.2 | N/A | 22.82 | 15.51 | 15.17 | 12.23 |
| 38 | 29.4 | 29.53 | 23.1 | 10.24 | 10.59 | 10.17 |
| 39 | 36.35 | 35.97 | 19.2 | 13.29 | 13.67 | 12.31 |
| 40 | 36.09 | 36.37 | 23.54 | 12.54 | 13.28 | 13.06 |
| Process | N/A | N/A | N/A | N/A | N/A | N/A |
| cont | | | | | | |
| Negative | N/A | N/A | N/A | N/A | N/A | N/A |

TABLE 21
Summary of the overall results of the Prospective Study

| PCR Ct Value | qRT-PCR | GSL LAMP |
|---|---|---|
| <20 | 3 | 3 |
| 20-24.99 | 20 | 20 |
| 25-29.99 | 46 | 46 |
| 30-34.99 | 31 | 31 |
| 35-36.99 | 11 | 11 |
| 37-39.99 | 6 | 6 |
| >40 | 1 | 4 |
| Presumptive positives | 3 | 0 |
| Negative | 0 | 0 |
| Total Positive samples | 118 | 121 |

The prospective study consisted of 121 positive SARS-CoV-2 samples that were collected from a local pathology laboratory. Presumptive positives are samples in which only 1 of the SARS-CoV-2 targets are positive in the assay. Using the GSL LAMP assay 3 samples that would have been presumptive positives by PCR were correctly called positive in the LAMP assay. Presumptive positive samples are generally not called positive thus the GSL LAMP gave 100% sensitivity (121/121) whereas PCR gave 97.5% sensitivity (118/121).

### Retrospective Study

In the Retrospective Study, 82 SARS-CoV-2 archived at −80° C. for over 18 months were diluted in UTM and split in two. Samples were then amplified with the GSL Real Time PCR and the Quadplex GSL modified RT-LAMP assays as described above. Presumptive positive samples, where only 1 gene is positive, are not included in the total numbers. There were samples still detected at Ct>40 with GSL LAMP. There was 100% concordance using with qRT-PCR using prospectively collected sample. The detection of samples with a Ct value >40 generally indicates the presence of only 1 or 2 copies of target sequence in the sample demonstrating the improved sensitivity of the GSL LAMP procedure. The data showed slightly reduced performance in the retrospective samples which may be caused by sample degradation and the larger size of the LAMP amplicon (250 bp) compared to qRT-PCR (<100 bp).

TABLE 22
Summary of the overall results of the Retrospective Study

| PCR Ct Value | qRT-PCR | GSL LAMP |
|---|---|---|
| 20-24.99 | 1 | 1 |
| 25-29.99 | 10 | 10 |
| 30-34.99 | 22 | 22 |
| 35-36.99 | 9 | 8 |
| 37-39.99 | 7 | 5 |
| >40 | 1 | 0 |
| Presumptive positives | 5 | 3 |
| Negative | 27 | 33 |
| Total Positive samples | 50 | 46 |

### Example 9: Comparison to Color Genomics N Gene Primers

The specificity of the Color (https://www.color.com) LAMP N primers were tested against a range of clinical extracts and no template controls. The N gene primers used were the ‘Color SARS-CoV-2 LAMP Diagnostic Assay’ primers which has been provided with an Emergency use Authorisation (EUA) by the FDA. For this experiment the primers were synthesised based on the sequences listed in Dudley D M, Newman C M, Weiler A M, Ramuta M D, Shortreed C G, Heffron A S, et al. (2020) Optimizing direct RT-LAMP to detect transmissible SARS-CoV-2 from primary nasopharyngeal swab samples. PLOS ONE 15 (12): e0244882, with the addition of a T7 tailed F3 primer. As can be seen from the data in Table 23 (below) after around 18 minutes false positive amplification curves were generated using the no template control (NTC) assays using the Color N gene primers. In contrast, using the T7 LAMP variant assay described herein no false positive signals were generated even after 25 minutes of amplification.

False positive amplification signals are a well-documented drawback of the LAMP method. As shown in Table 23, the T7 modified LAMP protocol reduces the incidence of false positives.

TABLE 23
Comparison of Color N gene assay vs Gene N gene T7 LAMP

| | |
|---|---|
| | Time |
| Sample | (min) |

Color N gene assay control

| | |
|---|---|
| 1 | 11.62 |
| 2 | 12.26 |
| 3 | N/A |
| 4 | 12.5 |
| 5 | 13.26 |
| 6 | 13.82 |
| 7 | 22.05 |
| 8 | 21.1 |
| 9 | 17.63 |
| 10 | 18.27 |
| 11 | 17.56 |
| 12 | 24.21 |
| 13 | 17.29 |
| 14 | 21.2 |
| 15 | N/A |
| 16 | N/A |
| 17 | 22.03 |
| 18 | 23.38 |
| 19 | 9.34 |
| 20 | N/A |
| 21 | 13.11 |
| 22 | 9.57 |
| 23 | 9.45 |
| 24 | 13.82 |
| 25 | N/A |
| 26 | 15.01 |
| 27 | 11.2 |
| 28 | 10.13 |
| 29 | 9.29 |
| 30 | N/A |
| 31 | 12.01 |
| 32 | 12.07 |
| 33 | 23.18 |
| 34 | 20.14 |
| 35 | 23.51 |
| 36 | 13.16 |
| NTC-1 | 20.62 |
| NTC-2 | 19.34 |
| NTC-3 | 18.06 |
| NTC-4 | 18.83 |

Color N gene T7 LAMP

| | |
|---|---|
| 1 | 10.21 |
| 2 | 11.65 |
| 3 | N/A |
| 4 | 11.81 |
| 5 | 11.49 |
| 6 | 12.11 |
| 7 | 23.31 |
| 8 | N/A |
| 9 | N/A |
| 10 | 12.4 |
| 11 | 16.11 |
| 12 | N/A |
| 13 | N/A |
| 14 | N/A |
| 15 | N/A |
| 16 | N/A |
| 17 | 20.65 |
| 18 | N/A |
| 19 | 8.1 |
| 20 | N/A |
| 21 | 11.35 |
| 22 | 8.42 |
| 23 | 8.38 |
| 24 | 11.78 |
| 25 | N/A |
| 26 | 12.49 |
| 27 | 9.85 |
| 28 | 9 |
| 29 | 8.07 |
| 30 | N/A |
| 31 | 10.43 |
| 32 | 11.11 |
| 33 | 13.03 |
| 34 | 12.26 |
| 35 | N/A |
| 36 | 11.02 |
| NTC-1 | N/A |
| NTC-2 | N/A |
| NTC-3 | N/A |
| NTC-4 | N/A |

The specificity of the LAMP As1e assay was tested against a range of SARS-CoV-2 clinical extracts, no template controls and positive influenza A samples. As can be seen from the data after around 15 minutes false positive amplification curves were generated using the control assay. In addition false positive signals were seen using previously tested Influenza A clinical samples. However, using the T7 LAMP variant assay no false positive signals were generated even after 20 minutes of amplification in both NTC and Influenza A samples. False positive amplification signals are a well-documented drawback of the LAMP method. The T7 modified LAMP protocol thus improves the utility of the LAMP method and results in an increase in the detection of low positive signals. In addition, samples 8, 10 and 29 were positive in the T7 lamp As1e assay after 6-11 minutes but negative in the control assay.

TABLE 24
Comparison of As1e gene assay vs As1e gene T7 LAMP

| | |
|---|---|
| Sample | Time |
| # | (min) |

As1e gene assay control

| | |
|---|---|
| 1 | 7.85 |
| 2 | 8.01 |
| 3 | 19.28 |
| 4 | 9.56 |
| 5 | 9.11 |
| 6 | 13.05 |
| 7 | 18.81 |
| 8 | 17.85 |
| 9 | 17.28 |
| 10 | 17.39 |
| 11 | 19.27 |
| 12 | 16.57 |
| 13 | N/A |
| 14 | 19.97 |
| 15 | 19.62 |
| 16 | 19.65 |
| 17 | 17.83 |
| 18 | 19.06 |
| 19 | 6.19 |
| 20 | 17.33 |
| 21 | 9.35 |
| 22 | 6.46 |
| 23 | 6.33 |
| 24 | 9.57 |
| 25 | 18.23 |
| 26 | 10.88 |
| 27 | 7.48 |
| 28 | 7.04 |
| 29 | N/A |
| 30 | 18.86 |
| 31 | 9.09 |
| 32 | 9.32 |
| 33 | 9.41 |
| 34 | 8.89 |
| 35 | 19.83 |
| 36 | 9.18 |
| NTC-1 | 15.11 |
| NTC-2 | 18.72 |
| NTC-3 | 15.89 |
| NTC-4 | 19.8 |
| Flu A-1 | 19.96 |
| Flu A-2 | 17.26 |
| Flu A-3 | 17.63 |
| Flu A-4 | 18.18 |
| Flu A-5 | 19.86 |
| Flu A-6 | 19.33 |
| Flu A-7 | N/A |
| Flu A-8 | 17.17 |

As1e gene T7 LAMP

| | |
|---|---|
| 1 | 8.64 |
| 2 | 9.66 |
| 3 | N/A |
| 4 | 9.89 |
| 5 | 9.33 |
| 6 | 10.14 |
| 7 | N/A |
| 8 | 11.63 |
| 9 | N/A |
| 10 | 11.11 |
| 11 | N/A |
| 12 | N/A |
| 13 | N/A |
| 14 | N/A |
| 15 | N/A |
| 16 | N/A |
| 17 | N/A |
| 18 | N/A |
| 19 | 6.5 |
| 20 | N/A |
| 21 | 10.13 |
| 22 | 7.09 |
| 23 | 6.92 |
| 24 | 9.04 |
| 25 | N/A |
| 26 | 11.6 |
| 27 | 8.5 |
| 28 | 7.23 |
| 29 | 6.63 |
| 30 | N/A |
| 31 | 9.06 |
| 32 | 9.04 |
| 33 | 13.02 |
| 34 | 9.99 |
| 35 | N/A |
| 36 | 9.81 |
| NTC-1 | N/A |
| NTC-2 | N/A |
| NTC-3 | N/A |
| NTC-4 | N/A |
| Flu A-1 | N/A |
| Flu A-2 | N/A |
| Flu A-3 | N/A |
| Flu A-4 | N/A |
| Flu A-5 | N/A |
| Flu A-6 | N/A |
| Flu A-7 | N/A |
| Flu A-8 | N/A |

### Example 10: Comparison to Known LAMP Reactions

The ability of a LAMP primer comprising a promoter for an RNA polymerase to improve a LAMP reaction was tested by adding a T7 promoter to a variety of published promoter sequences (see Table 25 for promoter sequences), specifically a T7 promoter was added to the F3 primers.

TABLE 25
Additional Primer sequences

| Primer name | Sequence | SEQ ID NO: |
|---|---|---|
| Color-ORF1a-F3 | TCCAGATGAGGATGAAGAAGA | 68 |
| Color-ORF1a-B3 | AGTCTGAACAACTGGTGTAAG | 69 |
| Color-ORF1a-FIP | AGAGCAGCAGAAGTGGCACAGGTGATTGTGAAGAAGAAGAG | 70 |
| Color-ORF1a-BIP | TCAACCTGAAGAAGAGCAAGAACTGATTGTCCTCACTGCC | 71 |
| Color-ORF1a-LF | CTCATATTGAGTTGATGGCTCA | 72 |
| Color-ORF1a-LB | ACAAACTGTTGGTCAACAAGAC | 73 |
| Gene-N-A-F3 | TGGCTACTACCGAAGAGCT | 74 |
| Gene-N-A-B3 | TGCAGCATTGTTAGCAGGAT | 75 |
| Gene-N-A-FIP | TCTGGCCCAGTTCCTAGGTAGTCCAGACGAATTCGTGGTGG | 76 |
| Gene-N-A-BIP | AGACGGCATCATATGGGTTGCACGGGTGCCAATGTGATCT | 77 |
| Gene-N-A-LF | GGACTGAGATCTTTCATTTTACCGT | 78 |
| Gene-N-A-LB | ACTGAGGGAGCCTTGAATACA | 79 |
| Yu-ORF1a-F3 | CCACTAGAGGAGCTACTGTA | 80 |
| Yu-ORF1a-B3 | TGACAAGCTACAACACGT | 81 |
| Yu-ORF1a-FIP | AGGTGAGGGTTTTCTACATCACTATATTGGAACAAGCAAATTC | 82 |
| | TATGG | |
| Yu-ORF1a-BIP | ATGGGTTGGGATTATCCTAAATGTGTGCGAGCAAGAACAAGT | 83 |
| | G | |
| Yu-ORF1a-LF | CAGTTTTTAACATGTTGTGCCAACC | 84 |
| Yu-ORF1a-LB | TAGAGCCATGCCTAACATGCT | 85 |
| El-Tholoth- | TGCTTCAGTCAGCTGATG | 86 |
| ORF1a-F3 | | |
| El-Tholoth- | TTAAATTGTCATCTTCGTCCTT | 87 |
| ORF1a-B3 | | |
| El-Tholoth- | TCAGTACTAGTGCCTGTGCCCACAATCGTTTTTAAACGGGT | 88 |
| ORF1a-FIP | | |
| El-Tholoth- | TCGTATACAGGGCTTTTGACATCTATCTTGGAAGCGACAACA | 89 |
| ORF1a-BIP | A | |
| El-Tholoth- | CTGCACTTACACCGCAA | 90 |
| ORF1a-LF | | |
| El-Tholoth- | GTAGCTGGTTTTGCTAAATTCC | 91 |
| ORF1a-LB | | |
| Lamb-ORF1a-F3 | TCCAGATGAGGATGAAGAAGA | 92 |
| Lamb-ORF1a-B3 | AGTCTGAACAACTGGTGTAAG | 93 |
| Lamb-ORF1a-FIP | AGAGCAGCAGAAGTGGCACAGGTGATTGTGAAGAAGAAGAG | 94 |
| Lamb-ORF1a-BIP | TCAACCTGAAGAAGAGCAAGAACTGATTGTCCTCACTGCC | 95 |
| Lamb-ORF1a-LF | CTCATATTGAGTTGATGGCTCA | 96 |
| Lamb-ORF1a-LB | ACAAACTGTTGGTCAACAAGAC | 97 |
| As1e-F3 | CGGTGGACAAATTGTCAC | 98 |
| As1e-F3 T7 | AATTCTAATACGACTCACTATAGGGCCCATAATCGGTGGACA | 99 |
| | AATTGTCAC | |
| As1e-B3 | CTTCTCTGGATTTAACACACTT | 100 |
| As1e-FIP | TCAGCACACAAAGCCAAAAATTTATTTTTCTGTGCAAAGGAAA | 101 |
| | TTAAGGAG | |
| As1e-BIP | TATTGGTGGAGCTAAACTTAAAGCCTTTTCTGTACAATCCCTT | 102 |
| | TGAGTG | |
| As1e-LF | TTACAAGCTTAAAGAATGTCTGAACACT | 103 |
| As1e-LB | TTGAATTTAGGTGAAACATTTGTCACG | 104 |

The Color-ORF1a primers in Table 26 are described in Color Genomics. SARS-CoV-2 LAMP Diagnostic Assay. 2020 https://www.color.com/wp-content/uploads/2020/05/LAMP-Diagnostic-Assay.pdf.

The Gene-N-A primers in Table 27 are described in Broughton J P, Deng X, Yu G, et al. CRISPR-Cas12-based detection of SARS-CoV-2. Nat Biotechnol. 2020 July; 38:870-874.

The Yu-ORF1a primers in Table 28 are described in Yu L, Wu S, Hao X, et al. Rapid Detection of COVID-19 Coronavirus Using a Reverse Transcriptional Loop-Mediated Isothermal Amplification (RT-LAMP) Diagnostic Platform. [letter]. Clin Chem 2020 Jul. 1; 66 (7): 975-977.

The in El-Tholoth-ORF1a in Table 29 are described in El-Tholoth M, Bau H H, Song J. A single and two-stage, closed tube, molecular test for the 2019 novel coronavirus (COVID-19) at home, clinic, and points of entry. ChemRxiv. 2020.

The Lamb-ORF1a primers in table 30 are described in Lamb L E, Bartolone S N, Ward E, Chancellor M B. Rapid detection of novel coronavirus/Severe Acute Respiratory Syndrome Coronavirus 2 (SARS-CoV-2) by reverse transcription-loop-mediated isothermal amplification. PLOS One. 2020; 15

As can be seen from Tables 26-30 the T7 LAMP assay results in improved performance in terms of sensitivity and a reduction in false positives.

TABLE 26
Color ORF1a vs Color ORF1a T7 LAMP

| | |
|---|---|
| | time |
| Sample# | (mins) |

Color OFR1a Control

| | |
|---|---|
| 1 | N/A |
| 2 | N/A |
| 3 | N/A |
| 4 | N/A |
| 5 | N/A |
| 6 | N/A |
| 7 | N/A |
| 8 | N/A |
| 9 | N/A |
| 10 | N/A |
| 11 | N/A |
| 12 | N/A |
| 13 | N/A |
| 14 | N/A |
| 15 | N/A |
| 16 | N/A |
| 17 | N/A |
| 18 | N/A |
| 19 | 19.12 |
| 20 | N/A |
| 21 | N/A |
| 22 | 19.99 |
| 23 | 19.72 |
| 24 | N/A |
| 25 | N/A |
| 26 | N/A |
| 27 | N/A |
| 28 | 19.35 |
| 29 | 18.34 |
| 30 | N/A |
| 31 | N/A |
| 32 | N/A |
| 33 | N/A |
| 34 | N/A |
| 35 | N/A |
| 36 | N/A |
| NTC-1 | N/A |
| NTC-2 | N/A |
| NTC-3 | N/A |
| NTC-4 | N/A |
| Flu A-1 | N/A |
| Flu A-2 | N/A |
| Flu A-3 | N/A |
| Flu A-4 | N/A |
| Flu A-5 | N/A |
| Flu A-6 | N/A |
| Flu A-7 | N/A |
| Flu A-8 | N/A |

Color OFR1a T7 LAMP

| | |
|---|---|
| 1 | 16.54 |
| 2 | 17.07 |
| 3 | N/A |
| 4 | N/A |
| 5 | 18.68 |
| 6 | 20.02 |
| 7 | 20.91 |
| 8 | N/A |
| 9 | N/A |
| 10 | 18.85 |
| 11 | N/A |
| 12 | N/A |
| 13 | N/A |
| 14 | N/A |
| 15 | N/A |
| 16 | N/A |
| 17 | N/A |
| 18 | N/A |
| 19 | 13.58 |
| 20 | N/A |
| 21 | 18.7 |
| 22 | 14.06 |
| 23 | 14.04 |
| 24 | 18.26 |
| 25 | N/A |
| 26 | 19.98 |
| 27 | 16.51 |
| 28 | 14.69 |
| 29 | 13.42 |
| 30 | N/A |
| 31 | 19.91 |
| 32 | 18.32 |
| 33 | N/A |
| 34 | 17.47 |
| 35 | N/A |
| 36 | 17 |
| NTC-1 | N/A |
| NTC-2 | N/A |
| NTC-3 | N/A |
| NTC-4 | N/A |
| Flu A-1 | N/A |
| Flu A-2 | N/A |
| Flu A-3 | N/A |
| Flu A-4 | N/A |
| Flu A-5 | N/A |
| Flu A-6 | N/A |
| Flu A-7 | N/A |
| Flu A-8 | N/A |

TABLE 27
Gene-N control vs Gene-N T7 LAMP

| | |
|---|---|
| | time |
| Sample# | (mins) |

Gene-N Control

| | |
|---|---|
| 1 | 20.13 |
| 2 | 19.7 |
| 3 | N/A |
| 4 | 20.13 |
| 5 | 21.01 |
| 6 | N/A |
| 7 | N/A |
| 8 | N/A |
| 9 | N/A |
| 10 | N/A |
| 11 | N/A |
| 12 | N/A |
| 13 | N/A |
| 14 | N/A |
| 15 | N/A |
| 16 | N/A |
| 17 | N/A |
| 18 | N/A |
| 19 | 15.55 |
| 20 | N/A |
| 21 | N/A |
| 22 | 16.07 |
| 23 | 16.03 |
| 24 | N/A |
| 25 | N/A |
| 26 | 21.74 |
| 27 | 18.95 |
| 28 | 16.87 |
| 29 | 15.55 |
| 30 | N/A |
| 31 | 19.09 |
| 32 | N/A |
| 33 | N/A |
| 34 | 19.49 |
| 35 | 21.02 |
| 36 | 20.75 |
| NTC-1 | N/A |
| NTC-2 | N/A |
| NTC-3 | N/A |
| NTC-4 | N/A |
| Flu A-1 | N/A |
| Flu A-2 | N/A |
| Flu A-3 | N/A |
| Flu A-4 | N/A |
| Flu A-5 | N/A |
| Flu A-6 | N/A |
| Flu A-7 | N/A |
| Flu A-8 | N/A |

Gene-N T7 LAMP

| | |
|---|---|
| 1 | 15.32 |
| 2 | 14.8 |
| 3 | N/A |
| 4 | 16.46 |
| 5 | 15.94 |
| 6 | 18.54 |
| 7 | N/A |
| 8 | N/A |
| 9 | N/A |
| 10 | 16.87 |
| 11 | N/A |
| 12 | N/A |
| 13 | N/A |
| 14 | N/A |
| 15 | N/A |
| 16 | N/A |
| 17 | N/A |
| 18 | N/A |
| 19 | 11.61 |
| 20 | 16.95 |
| 21 | N/A |
| 22 | 12.19 |
| 23 | 12.22 |
| 24 | 15.04 |
| 25 | N/A |
| 26 | N/A |
| 27 | 14.26 |
| 28 | 12.73 |
| 29 | 11.59 |
| 30 | 20.23 |
| 31 | 14.63 |
| 32 | 14.98 |
| 33 | 16.79 |
| 34 | 17.14 |
| 35 | N/A |
| 36 | 15.57 |
| NTC-1 | N/A |
| NTC-2 | N/A |
| NTC-3 | N/A |
| NTC | N/A |
| FluA-1 | N/A |
| Flu A-2 | N/A |
| Flu A-3 | N/A |
| Flu A-4 | N/A |
| Flu A-5 | N/A |
| Flu A-6 | N/A |
| Flu A-7 | N/A |
| Flu A-8 | N/A |

TABLE 28
Yu et al control vs Yu et al T7 LAMP

| | |
|---|---|
| | time |
| Sample# | (mins) |

Yu et al Control

| | |
|---|---|
| 1 | 10.81 |
| 2 | 13.01 |
| 3 | N/A |
| 4 | N/A |
| 5 | 12.53 |
| 6 | 16.6 |
| 7 | 23.62 |
| 8 | N/A |
| 9 | N/A |
| 10 | 25.77 |
| 11 | N/A |
| 12 | 27.38 |
| 13 | 18.6 |
| 14 | 22.73 |
| 15 | N/A |
| 16 | N/A |
| 17 | N/A |
| 18 | 28.95 |
| 19 | 9.03 |
| 20 | N/A |
| 21 | 24.73 |
| 22 | 9.33 |
| 23 | 9.25 |
| 24 | 13.03 |
| 25 | 27.67 |
| 26 | 23.24 |
| 27 | 10.96 |
| 28 | 9.64 |
| 29 | 9.08 |
| 30 | 27.53 |
| 31 | 12.84 |
| 32 | 12.13 |
| 33 | 12.92 |
| 34 | 11.14 |
| 35 | 26.94 |
| 36 | 11.92 |
| NTC-1 | 20.09 |
| NTC-2 | 21.06 |
| NTC-3 | 21.81 |
| NTC-4 | 26.22 |
| Flu A-1 | 29.37 |
| Flu A-2 | 24.64 |
| Flu A-3 | 24.45 |
| Flu A-4 | N/A |
| Flu A-5 | N/A |
| Flu A-6 | N/A |
| Flu A-7 | N/A |
| Flu A-8 | 28.99 |

Yu et al T7 LAMP

| | |
|---|---|
| 1 | 9.62 |
| 2 | 11.77 |
| 3 | 29.28 |
| 4 | 13.53 |
| 5 | 11.55 |
| 6 | 13.02 |
| 7 | N/A |
| 8 | N/A |
| 9 | N/A |
| 10 | N/A |
| 11 | 24.62 |
| 12 | 25.13 |
| 13 | N/A |
| 14 | N/A |
| 15 | N/A |
| 16 | N/A |
| 17 | N/A |
| 18 | N/A |
| 19 | 8.06 |
| 20 | N/A |
| 21 | 28.34 |
| 22 | 8.36 |
| 23 | 8.25 |
| 24 | 11.68 |
| 25 | N/A |
| 26 | N/A |
| 27 | 27.65 |
| 28 | N/A |
| 29 | 25.31 |
| 30 | 18.09 |
| 31 | 15.47 |
| 32 | N/A |
| 33 | 28.02 |
| 34 | 12.9 |
| 35 | N/A |
| 36 | 16.01 |
| NTC-1 | N/A |
| NTC-2 | N/A |
| NTC-3 | N/A |
| NTC-4 | N/A |
| Flu A-1 | N/A |
| Flu A-2 | N/A |
| Flu A-3 | N/A |
| Flu A-4 | N/A |
| Flu A-5 | N/A |
| Flu A-6 | N/A |
| Flu A-7 | N/A |
| Flu A-8 | N/A |

TABLE 29
El-Tholoth Control vs El-Tholoth T7 LAMP

| | |
|---|---|
| | time |
| Sample# | (mins) |

El-Tholoth Control

| | |
|---|---|
| 1 | 12.09 |
| 2 | 13.29 |
| 3 | N/A |
| 4 | 14.2 |
| 5 | 12.98 |
| 6 | 14.02 |
| 7 | N/A |
| 8 | N/A |
| 9 | N/A |
| 10 | 16.28 |
| 11 | N/A |
| 12 | N/A |
| 13 | N/A |
| 14 | N/A |
| 15 | N/A |
| 16 | N/A |
| 17 | 14.26 |
| 18 | N/A |
| 19 | 10.19 |
| 20 | N/A |
| 21 | N/A |
| 22 | 10.61 |
| 23 | 9.37 |
| 24 | 13.34 |
| 25 | N/A |
| 26 | 14.68 |
| 27 | 12.09 |
| 28 | 10.87 |
| 29 | 9.99 |
| 30 | N/A |
| 31 | 12.64 |
| 32 | 12.85 |
| 33 | 13.59 |
| 34 | 15.48 |
| 35 | N/A |
| 36 | 13.96 |
| NTC-1 | N/A |
| NTC-2 | N/A |
| NTC-3 | N/A |
| NTC-4 | N/A |
| Flu A-1 | N/A |
| Flu A-2 | N/A |
| Flu A-3 | N/A |
| Flu A-4 | N/A |
| Flu A-5 | N/A |
| Flu A-6 | N/A |
| Flu A-7 | N/A |
| Flu A-8 | N/A |

El-Tholoth T7 LAMP

| | |
|---|---|
| 1 | 10.72 |
| 2 | 11.43 |
| 3 | N/A |
| 4 | 12.68 |
| 5 | 11.63 |
| 6 | 14.7 |
| 7 | N/A |
| 8 | N/A |
| 9 | N/A |
| 10 | N/A |
| 11 | N/A |
| 12 | N/A |
| 13 | N/A |
| 14 | N/A |
| 15 | N/A |
| 16 | N/A |
| 17 | N/A |
| 18 | N/A |
| 19 | 8.92 |
| 20 | N/A |
| 21 | 12.25 |
| 22 | 9.16 |
| 23 | 9 |
| 24 | 11.99 |
| 25 | N/A |
| 26 | N/A |
| 27 | 10.5 |
| 28 | 9.48 |
| 29 | 8.59 |
| 30 | 14.66 |
| 31 | 11.4 |
| 32 | 10.63 |
| 33 | 13.47 |
| 34 | 12.09 |
| 35 | N/A |
| 36 | 11.58 |
| NTC-1 | N/A |
| NTC-2 | N/A |
| NTC-3 | N/A |
| NTC-4 | N/A |
| Flu A-1 | N/A |
| Flu A-2 | N/A |
| Flu A-3 | N/A |
| Flu A-4 | N/A |
| Flu A-5 | N/A |
| Flu A-6 | N/A |
| Flu A-7 | N/A |
| Flu A-8 | N/A |

TABLE 30
LAMB Control vs LAMB T7 LAMP

| | |
|---|---|
| | time |
| Sample# | (mins) |

LAMB Control

| | |
|---|---|
| 1 | N/A |
| 2 | N/A |
| 3 | N/A |
| 4 | N/A |
| 5 | N/A |
| 6 | N/A |
| 7 | N/A |
| 8 | N/A |
| 9 | N/A |
| 10 | N/A |
| 11 | N/A |
| 12 | N/A |
| 13 | N/A |
| 14 | N/A |
| 15 | N/A |
| 16 | N/A |
| 17 | N/A |
| 18 | N/A |
| 19 | 19.84 |
| 20 | N/A |
| 21 | N/A |
| 22 | 20.42 |
| 23 | 20.51 |
| 24 | N/A |
| 25 | Not |
| | Done |
| 26 | Not |
| | Done |
| 27 | Not |
| | Done |
| 28 | Not |
| | Done |
| 29 | Not |
| | Done |
| 30 | Not |
| | Done |
| 31 | Not |
| | Done |
| 32 | Not |
| | Done |
| 33 | N/A |
| 34 | N/A |
| 35 | N/A |
| 36 | N/A |
| NTC-1 | N/A |
| NTC-2 | N/A |
| NTC-3 | N/A |
| NTC-4 | N/A |
| Flu A-1 | N/A |
| Flu A-2 | N/A |
| Flu A-3 | N/A |
| Flu A-4 | N/A |
| Flu A-5 | N/A |
| Flu A-6 | N/A |
| Flu A-7 | N/A |
| Flu A-8 | N/A |

LAMB T7 LAMP

| | |
|---|---|
| 1 | 17.25 |
| 2 | 18.04 |
| 3 | N/A |
| 4 | 18.73 |
| 5 | 18.88 |
| 6 | 20.01 |
| 7 | N/A |
| 8 | N/A |
| 9 | N/A |
| 10 | 20.13 |
| 11 | N/A |
| 12 | N/A |
| 13 | N/A |
| 14 | N/A |
| 15 | N/A |
| 16 | N/A |
| 17 | N/A |
| 18 | N/A |
| 19 | 12.97 |
| 20 | N/A |
| 21 | N/A |
| 22 | 13.76 |
| 23 | 13.66 |
| 24 | 17.45 |
| 25 | Not |
| | Done |
| 26 | Not |
| | Done |
| 27 | Not |
| | Done |
| 28 | Not |
| | Done |
| 29 | Not |
| | Done |
| 30 | Not |
| | Done |
| 31 | Not |
| | Done |
| 32 | Not |
| | Done |
| 33 | N/A |
| 34 | N/A |
| 35 | N/A |
| 36 | 17.49 |
| NTC-1 | N/A |
| NTC-2 | N/A |
| NTC-3 | N/A |
| NTC-4 | N/A |
| Flu A-1 | N/A |
| Flu A-2 | N/A |
| Flu A-3 | N/A |
| Flu A-4 | N/A |
| Flu A-5 | N/A |
| Flu A-6 | N/A |
| Flu A-7 | N/A |
| Flu A-8 | N/A |

### Example 11. Comparison of T7 LAMP to Real-Time PCR

Table 31 shows the results of 28 randomly selected previously tested SARS COV-2 samples. The samples were diluted at least 10 fold prior to testing due to limited availability. As can be seen the T7-LAMP procedure is now approaching the sensitivity of the “gold-standard” PCR method.

TABLE 31
T7 LAMP vs Real-time PCR

| | | Real-time PCR | | |
|---|---|---|---|---|
| | T7-LAMP | SARS-Cov-2 | SARS-Cov-2 | |
| Sample | Minutes | M-gene | N-gene | 12S Control |
| 1 | N/A | N/A | N/A | 26.91 |
| 2 | N/A | 42.34 | 37.08 | 28.13 |
| 3 | N/A | N/A | N/A | 26.2 |
| 4 | N/A | N/A | N/A | 28.27 |
| 5 | 9.9 | 29.06 | 27.43 | 27.94 |
| 6 | 12.45 | 35.74 | 33.8 | 28.25 |
| 7 | 11.88 | 34.24 | 32.24 | 31.32 |
| 8 | 10.11 | 30.89 | 29.2 | 27.7 |
| 9 | 11.94 | 31.81 | 30.29 | 24.08 |
| 10 | N/A | N/A | N/A | 39.62 |
| 11 | N/A | N/A | N/A | 27.11 |
| 12 | 11.22 | 33.8 | 32.19 | 27.51 |
| 13 | 14.04 | 37.04 | 36.5 | 27.52 |
| 14 | 12.51 | 35.13 | 33.9 | 29.84 |
| 15 | 12.71 | 40.95 | 38.04 | 26.82 |
| 16 | 11.33 | 34.27 | 32.48 | 29.27 |
| 17 | 11.3 | 33.62 | 32.2 | 26.16 |
| 18 | 25.64 | N/A | N/A | 27.39 |
| 19 | 13.13 | 40.86 | 38.64 | 29.28 |
| 20 | N/A | N/A | N/A | N/A |
| 21 | N/A | N/A | N/A | 31.16 |
| 22 | 9.56 | 27.41 | 26.05 | 27.49 |
| 23 | 11.15 | 32.69 | 31.13 | 34.96 |
| 24 | N/A | N/A | N/A | 36.99 |
| 25 | 12.88 | 36.26 | 35.12 | 33.79 |
| 26 | 9.15 | 27.15 | 25.59 | 28.21 |
| 27 | 24.42 | N/A | N/A | 29.11 |
| 28 | N/A | N/A | N/A | 32.17 |

### Example 12. Extraction Free LAMP

80 clinical samples from the Prospective Study were diluted with extraction free buffer containing 400 mM GuHCl and proteinase K (7.5 μL of buffer and 12.5 μL of sample). The samples were then heated at 80° C. for 10 minutes then added directly to the GSL LAMP reaction containing modified primers for the Lateral Flow devise. Positive signals were seen 2-3 minutes after closing the detection chamber. The advantages of the extraction free LAMP method are that it only requires the use of a heat block and results may be obtained within 30 minutes, and it does not require the extraction of RNA or DNA, therefore it is particularly useful for resource-limited settings. The data also show that samples were still positive even with Ct values >35 but negative at Ct>37.

TABLE 32
Summary of results generated with extraction free LAMP

| | SP012/RP012 | Extraction Free |
|---|---|---|
| Ct Value | PCR Positive | Lateral Flow Positive |
| <24.99 | 5 | 5 |
| 25-29.99 | 32 | 32 |
| 30-34.99 | 25 | 24 |
| 35-36.99 | 9 | 5 |
| 37-39.99 | 6 | 0 |
| >40 | 1 | 0 |
| Presumptive positives | 2 | 0 |
| Total positives | 78 | 66 |

### Example 13. Samples

Further details of the samples identified in the examples above are provided in Table 33. Samples with the same description but different sample numbers (e.g. Clinical sample, Influenza A positive) are from different patients or sources.

TABLE 33
Sample details

| Sample | Description |
|---|---|
| Table 6. | Eight previously qPCR tested Influenza A clinical |
| | samples (not typed) obtained from a local hospital |
| Table 7. | 2021 QCMD SARS-COV-2 challenge C1B samples 1-5 |
| Table 8 | qPCR quantified SARS-COV-2 culture dilutions with |
| | copy number indicated obtained from a local hospital. |
| Table 9 | Same samples as used in table 8. |
| Table 10 | Same samples as used in table 8 |
| Table 11* | Eight randomly selected diluted SARS-COV-2 clinical |
| | samples obtained from a local pathology laboratory. |
| Table 12 | Same samples as used in table 11 |
| Table 14 | Samples were obtained from a local pathology laboratory. |
| Table 16 | Samples were obtained from a local pathology laboratory. |
| Table 17 | Samples were obtained from a local pathology laboratory. |
| Table 18 | Samples were obtained from a local pathology laboratory. |
| Table 20 | A hundred and twenty one SARS-COV-2 positive samples |
| | were freshly collected from a local hospital. |
| Table 22 | Eighty two SARS-COV-2 positive samples were obtained |
| | from −80° C. storage archived for over 18 months. |
| Table 23* | Thirty-six randomly selected diluted SARS-COV-2 |
| | clinical samples obtained from a local pathology laboratory. |
| Table 24 | Same samples as used in table 13.. |
| Table 26 | Same samples as used in table 13. |
| Table 27 | Same samples as used in table 13. |
| Table 28 | Same samples as used in table 13. |
| Table 29 | Same samples as used in table 13. |
| Table 30 | Same samples as used in table 13. |
| Table 31* | Twenty-eight randomly selected diluted SARS-COV-2 |
| | clinical samples obtained from a local pathology laboratory. |

*The dilution factor was at least 10 fold compared to the normal testing concentration.

### Example 14. T7 Placement Position

As described above, LAMP reaction with LAMP primers F3 and B3 synthesised with either T7 or SP6 tails (T7 or SP6 promoters) showed improved amplification sensitivity and efficiency compared to the control LAMP reaction. As shown in Table 6, data using the tailed primers resulted in both an increase in the number of positive samples detected and a reduction in the time to result thus demonstrating that both F3 and B3 outer primers can be used for RNA polymerase promoter placement.

Upon additional testing, the inventors found that mixtures of F3-T7 and B3-T7 did not improve sensitivity of the LAMP assay further and on testing a larger number of samples it was found that overall F3-T7 performed slightly better than B3-T7.

Table 34 shows the effect of placement of the T7 promoter on various LAMP primers. The data show the optimal placement for the T7 tail (at the 5′ end of the primer sequence) is on the outer F3 primer set. When the T7 tail is placed on the inner FIP primer there is essentially no difference in time to result using the RdRP (a SARS-CoV-2 gene) region 1 primer set, and with the RdRP primer set 2 the reaction is substantially inhibited. When the T7 tail is place on the internal Loop LF primer, there is very little difference when compared to the control reaction without the T7 tail. In contrast, when the T7 tail is placed on the F3 (outer primer) there is a decrease in the time to result and also an increase in positivity for the RdRP region 1 (F3-T7 results in bold).

TABLE 34
Comparison of LAMP primers and T7 tail placement

| | | | RdRP Region 1 | | | RdRP Region 2 | | |
|---|---|---|---|---|---|---|---|---|
| Sample# | LAMP | F3-T7 | FIP-T7 | LF-T7 | LAMP | F3-T7 | FIP-T7 | LF-T7 |
| 1 | 25.08 | 19.62 | 24.55 | 32.4 | 30.85 | 31.1 | N/A | 31.26 |
| 2 | 19.59 | 16.98 | 20.42 | 18.54 | 25.21 | 22.1 | 31.16 | 24.18 |
| 3 | N/A | 20.15 | 32.56 | 20.64 | 27.65 | 28.65 | 38.89 | N/A |
| 4 | 19.17 | 17.17 | 20.36 | 17.73 | 24.57 | 21.36 | 29.67 | 23.21 |
| 5 | 20.48 | 17.53 | 22.43 | 19.4 | 26.02 | 22.26 | 32.73 | 25.07 |
| 6 | N/A | N/A | N/A | N/A | N/A | N/A | N/A | N/A |
| 7 | 19.78 | 16.85 | 19.69 | 18.24 | 24 | 21.06 | 30.14 | 23.47 |
| 8 | N/A | N/A | N/A | N/A | N/A | N/A | N/A | N/A |

### Example 15. Neisseria gonorrhoea LAMP

LAMP reaction as described herein can also be used for bacterial detection. Table 35 shows the results of the GSL LAMP when used to detect the presence of N. gonorrhoea. LAMP primers were designed to a unique region of the 16S rRNA and amplified as usual (Table 36). As can be seen from the data, as few as 5-10 copies can be detected in the LAMP amplification reaction (Table 35).

TABLE 35
Results of N. gonorrohoea LAMP reaction

| | Time (mins) | |
|---|---|---|
| Copies/LAMP | Repl #1 | Repl #2 |
| 400 | 16.13 | 16.76 |
| 200 | 16.21 | 16.36 |
| 100 | 15.39 | 15.65 |
| 50 | 16.98 | 17.54 |
| 25 | 19.32 | 19.93 |
| 10 | 25.49 | 18.58 |
| 5 | 16.2 | N/A |
| 1 | N/A | N/A |
| NTC #1 | N/A | N/A |
| NTC #2 | N/A | N/A |
| NTC #3 | N/A | N/A |

TABLE 36
Primer sets used in N. gonorrohoea LAMP reaction

| Primer | | |
|---|---|---|
| Name | Sequence | SEQ ID NO: |
| NG-4 F3 | GCGGTGGATGATGTGGATT | 105 |
| NG-4 F3-T7 | AATTCTAATACGACTCACTATAGGGCCCATAATGCGGTGGA | 106 |
| | TGATGTGGATT | |
| NG-4 B3 | CCGGCAGTCTCATTAGAGTG | 107 |
| NG-4 B3-T7 | AATTCTAATACGACTCACTATAGGGCCCATAATCCGGCAGT | 108 |
| | CTCATTAGAGTG | |
| NG-4 FIP | CTCCTCCGTCTCCGGAGGATTCGATGCAACGCGAAGAACCT | 109 |
| NG-4 BIP | TCGTCAGCTCGTGTCGTGAGATCCCAACCGAATGATGGCA | 110 |
| NG-4 LF | CGCACATGTCAAAACCAGGTA | 112 |
| NG-4 LB | GTTGGGTTAAGTCCCGCAA | 113 |

TABLE 37
Cross-reactivity studies using N. gonorrohoea LAMP reaction

| | Cross Reactivity Panel | | | | | | |
|---|---|---|---|---|---|---|---|
| Organism | Ct | Organism | Ct | Organism | Ct | Organism | Ct |
| N. gonorrhoeae | 16.06 | C. jejunii | N/A | H3N2 | N/A | Dengue-1 | N/A |
| M. genitalium | N/A | C. difficile | N/A | H1N1 | N/A | Dengue-2 | N/A |
| B. pertussis | N/A | EHEC | N/A | Influenza B | N/A | Dengue-3 | N/A |
| C. pneumoniae | N/A | Salmonella | N/A | PIF-1 | N/A | Dengue-4 | N/A |
| C. psittaci | N/A | Shigella | N/A | PIF-2 | N/A | Zika | N/A |
| C. burrneti | N/A | Yersinia | N/A | PIF-3 | N/A | Chikungunya | N/A |
| L. pneumoniae | N/A | Adenovirus | N/A | RSV-A | N/A | HSV-1 | N/A |
| M. pneumoniae | N/A | Coronavirus 229E | N/A | RSV-B | N/A | HSV-2 | N/A |

Table 37 shows the results of cross-reactivity studies using the N. gonorrhoea LAMP reaction. No cross reactivity was observed with any of the bacterial or viral non-target organisms tested.

It will be appreciated by persons skilled in the art that numerous variations and/or modifications may be made to the invention as shown in the specific embodiments and examples above without departing from the spirit or scope of the invention as broadly described. The present embodiments are, therefore, to be considered in all respects as illustrative and not restrictive. In particular, it is to be understood that one skilled in the art, given the benefit of this disclosure, will be able to identify, select, optimize or modify suitable conditions and/or parameters for using the methods and kits in accordance with the principles of the invention, suitable for these and other types of applications. The precise use, choice of reagents, choice of variables such as concentration, volume, incubation time, incubation temperature, and the like may depend in large on the particular application for which it is intended.

1. A method of reducing the incidence of false positives, increasing efficiency, and/or increasing the sensitivity of loop-mediated isothermal amplification (LAMP) of a target nucleic acid comprising:
a) combining:
i) a plurality of LAMP primers for a target nucleic acid, wherein the plurality of LAMP primers comprises a F3 primer and a B3 primer, wherein both the F3 and B3 primer or the F3 primer comprises a promoter for an RNA polymerase at the 5′ end;
ii) dNTPs;
iii) a strand-displacing DNA polymerase;
iv) a reverse transcriptase;
v) an RNA polymerase; and
vi) rNTPs; and
vii) optionally a nucleic acid binding dye or probe,
to form a LAMP reagent mix,
b) incubating the LAMP reagent mix with the target nucleic acid under conditions suitable for amplification of the target nucleic acid with increased efficiency and/or sensitivity compared to the same amplification performed in the absence of the promoter, RNA polymerase and rNTPs.

2. The method of claim 1, wherein the probe is a fluorescent probe specific for the target.

3. The method of claim 1 or 2, wherein the promoter sequence is selected from a T7 promoter of SEQ ID NO. 1, a T3 promoter of SEQ ID NO. 2-4, and a SP6 promoter of SEQ ID NO. 5-6.

4. The method of any one of claim 1 to 3, wherein the RNA polymerase is selected from a T7 RNA polymerase, a T3 RNA polymerase and a SP6 RNA polymerase.

5. The method of any one of claims 1 to 4, wherein the concentration of the rNTPs is about 0.25 mM to about 2.5 mM and the concentration of the RNA polymerase is about 7.5 units per reaction to about 50 units per reaction.

6. The method of any one of claims 1 to 5, wherein the LAMP reagent mix comprises guanidinium hydrochloride (GuHCl).

7. The method of claim 6, wherein the GuHCl is at a concentration of at least about 30 mM.

8. The method of any one of claims 1 to 7, wherein the target nucleic acid is bisulphite treated.

9. The method of any one of claims 1 to 8, wherein the plurality of LAMP primers further comprises a FIP primer, a BIP primer, a LF primer and a LB primer.

10. The method of claim 9 wherein the FIP primer, the BIP primer, the LF primer and the LB primer do not comprise a promoter for an RNA polymerase.

11. The method of claim 9 or 10, wherein both the F3 primer and the B3 primer comprise a promoter sequence at the 5′ end, and wherein the promoter sequence is selected from a T7 promoter of SEQ ID NO. 1, a T3 promoter of SEQ ID NO. 2-4, and a SP6 promoter of SEQ ID NO. 5-6.

12. The method of claim 9 or 10, wherein the F3 primer comprises a promoter sequence at the 5′ end, and wherein the promoter sequence is selected from a T7 promoter of SEQ ID NO. 1, a T3 promoter of SEQ ID NO. 2-4, and a SP6 promoter of SEQ ID NO. 5-6.

13. The method of any one of claims 1 to 12, wherein the target nucleic acid is an RNA or DNA specific to a pathogen.

14. The method of claim 13, wherein the pathogen is selected from a bacterium, virus, fungus and parasite.

15. The method of claim 14, wherein the bacterium is selected from Salmonella spp, Bordetella spp, Campylobacter spp, Clostridium spp, Chlamydia spp, Chlamydophila spp, Listeria spp, Lymphogranuloma spp, Shigella spp, Neisseria spp, Staphylococcus spp, Streptococcus spp, Listeria spp, Leishmania spp, Bacillus spp, Borrelia spp, Rickettsia spp, Corynebacterium spp, Gardnerella spp, Haemophilus spp, Escherichia spp, Helicobacter spp, Klebsiella spp, Legionella spp, Mycobacterium spp, Mycoplasma spp; Moraxella spp, Pasteurella spp, Pneumocystis spp, Pseudomonas spp, Treponema spp, Ureaplasma spp, Vibrio sp, Aeromonas spp, and Yersinia spp.

16. The method of claim 14, wherein the virus is selected from a paraechovirus, rabies virus, measles virus, mumps virus, rubella virus, togaviridae, polyomavirus, papillomavirus, hepadnavirus, poxvirus, adenovirus, picornavirus, hepevirus, calicivirus, reovirus, retrovirus orthomyxovirus, paramyxovirus, coronavirus, Ebolavirus, Filoviridae, Flaviviridae, Rhabdoviridae, Bunyavirales, Arenaviridae, Hantaviridae and deltavirus.

17. The method of claim 16, wherein the coronavirus is Coronavirus HKU-1, Coronavirus OC43, Coronavirus NL63/229E, or SARS-CoV-2.

18. The method of claim 14, wherein the parasite is selected from Blastocystis spp, Giardia spp, Cryptosporidium spp, Cyclospora spp, Blastocystis spp, Dientamoeba spp, Entamoeba spp, Cryptococcus spp, Enterocytozoon spp, Encephalitozoon spp, Babesia spp, Leishmania spp, Schistosoma spp, Trypanosoma spp, Trichomonas spp, Treponema spp, and Plasmodium spp.

19. The method of claim 14, wherein the fungus is selected from Aspergillus spp, Candida spp, Histoplasma spp, Fusarium spp, Pneumocystis spp, Paracoccoides spp, Coccidioides spp, and Scedosporium spp.

20. The method of any one of claims 1 to 19, wherein the sensitivity of the LAMP reaction is increased by at least about 10% to about 30%.

21. The method of any one of claims 1 to 20, wherein the efficiency of the LAMP reaction is increased by at least about 5% to about 20%.

22. The method of any one of claims 1 to 21, wherein the loop-mediated isothermal amplification (LAMP) is a multiplex reaction and wherein the reaction comprises in step a) combining a second plurality of LAMP primers for a second target nucleic acid wherein the second plurality of LAMP primers comprises a F3 primer comprising a promoter for an RNA polymerase at the 5′ end.

23. A plurality of LAMP primers for a target nucleic acid, wherein the LAMP primers comprise a F3 primer and a B3 primer each comprising a promoter for an RNA polymerase at the 5′ end, or a F3 primer comprising a promoter for an RNA polymerase at the 5′ end, and at least one sequence complementary to the target nucleic acid.

24. A kit for detecting a target nucleic acid using a loop-mediated isothermal amplification (LAMP) reaction, the kit comprising
i) a plurality of LAMP primers for the target nucleic acid, wherein the plurality of LAMP primers comprises a F3 primer and a B3 primer, wherein both the F3 and B3 primer or the F3 primer comprises a promoter for an RNA polymerase at the 5′end;
ii) dNTPs;
iii) a strand-displacing DNA polymerase;
iv) a reverse transcriptase;
v) an RNA polymerase;
vi) rNTPs; and
vii) optionally a nucleic acid binding dye or probe.

25. The kit of claim 24, wherein the probe is a fluorescent probe specific for the target.

26. The kit of claim 24 or 25, wherein the target nucleic acid is an RNA or DNA specific pathogen.

27. The kit of any one of claims 24 to 26, wherein the plurality of LAMP primers further comprises a FIP primer, a BIP primer, a LF primer and a LB primer.

28. The kit of claim 27, wherein the FIP primer, the BIP primer, the LF primer and the LB primer do not comprise a promoter for an RNA polymerase.

29. The kit of any one of claims 24 to 28, wherein the loop-mediated isothermal amplification (LAMP) is a multiplex reaction.

30. The kit of claim 29, further comprising a second plurality of LAMP primers for a second target nucleic acid wherein the second plurality of LAMP primers comprises a F3 primer comprising a promoter for an RNA polymerase at the 5′ end.


SEQUENCE LISTING (112 sequences)

SEQ ID NO: 1 - DNA, 30 bp, unidentified
  source 1..30: mol_type = other DNA
aattctaatacgactcactatagggataat

SEQ ID NO: 2 - DNA, 27 bp, unidentified
  source 1..27: mol_type = other DNA
aattcattaaccctcactaaagataat

SEQ ID NO: 3 - DNA, 30 bp, unidentified
  source 1..30: mol_type = other DNA
aattcattaaccctcactaaagggaataat

SEQ ID NO: 4 - DNA, 34 bp, unidentified
  source 1..34: mol_type = other DNA
aattcgcgcgaaattaaccctcactaaagataat

SEQ ID NO: 5 - DNA, 34 bp, unidentified
  source 1..34: mol_type = other DNA
aattccatacgatttaggtgacactatagataat

SEQ ID NO: 6 - DNA, 26 bp, unidentified
  source 1..26: mol_type = other DNA
aattcatttaggtgacactatagggg

SEQ ID NO: 7 - DNA, 18 bp, unidentified
  source 1..18: mol_type = other DNA
taggcttgatgtggctca

SEQ ID NO: 8 - DNA, 18 bp, unidentified
  source 1..18: mol_type = other DNA
acagctccgattacgagt

SEQ ID NO: 9 - DNA, 45 bp, unidentified
  source 1..45: mol_type = other DNA
ctggattgaatgaccacatggaagctacttcattgcttctttcag

SEQ ID NO: 10 - DNA, 43 bp, unidentified
  source 1..43: mol_type = other DNA
aactaacattcttctcaacgtgcctcactttctagaagcggtc

SEQ ID NO: 11 - DNA, 17 bp, unidentified
  source 1..17: mol_type = other DNA
gcgtacgcgcaaacagt

SEQ ID NO: 12 - DNA, 21 bp, unidentified
  source 1..21: mol_type = other DNA
actccatggcactattctgac

SEQ ID NO: 13 - DNA, 19 bp, unidentified
  source 1..19: mol_type = other DNA
tggctactaccgaagagct

SEQ ID NO: 14 - DNA, 20 bp, unidentified
  source 1..20: mol_type = other DNA
tgcagcattgttagcaggat

SEQ ID NO: 15 - DNA, 41 bp, unidentified
  source 1..41: mol_type = other DNA
tctggcccagttcctaggtagtccagacgaattcgtggtgg

SEQ ID NO: 16 - DNA, 40 bp, Una virus
  source 1..40: mol_type = other DNA
agacggcatcatatgggttgcacgggtgccaatgtgatct

SEQ ID NO: 17 - DNA, 24 bp, unidentified
  source 1..24: mol_type = other DNA
gaaataccatcttggactgagatc

SEQ ID NO: 18 - DNA, 20 bp, unidentified
  source 1..20: mol_type = other DNA
actgagggagccttgaatac

SEQ ID NO: 19 - DNA, 20 bp, unidentified
  source 1..20: mol_type = other DNA
aatacgactcactatagggc

SEQ ID NO: 20 - DNA, 22 bp, unidentified
  source 1..22: mol_type = other DNA
ttcagatttttaacacgagagt

SEQ ID NO: 21 - DNA, 49 bp, unidentified
  source 1..49: mol_type = other DNA
cgaaagcaagaaaaagaagtacgctccaacttatgtactcattcgtttc

SEQ ID NO: 22 - DNA, 44 bp, unidentified
  source 1..44: mol_type = other DNA
ttgctagttacactagccatccttaggttttacaagactcacgt

SEQ ID NO: 23 - DNA, 24 bp, unidentified
  source 1..24: mol_type = other DNA
actattaacgtacctgtctcttcc

SEQ ID NO: 24 - DNA, 18 bp, unidentified
  source 1..18: mol_type = other DNA
actgcgcttcgattgtgt

SEQ ID NO: 25 - DNA, 25 bp, unidentified
  source 1..25: mol_type = other DNA
gttatattgtttttatagattagtt

SEQ ID NO: 26 - DNA, 20 bp, unidentified
  source 1..20: mol_type = other DNA
acaattttattaccatcaat

SEQ ID NO: 27 - DNA, 44 bp, unidentified
  source 1..44: mol_type = other DNA
acctaatttaacatataataaaccaatgagtgtgtttaagtatt

SEQ ID NO: 28 - DNA, 58 bp, unidentified
  source 1..58: mol_type = other DNA
ggaattttattaggagatgttataattgtttataaataaaaatacattaacattaacc

SEQ ID NO: 29 - DNA, 16 bp, unidentified
  source 1..16: mol_type = other DNA
cacataaccatttcac

SEQ ID NO: 30 - DNA, 22 bp, unidentified
  source 1..22: mol_type = other DNA
gttaatagtgtttttaatattt

SEQ ID NO: 31 - DNA, 19 bp, unidentified
  source 1..19: mol_type = other DNA
tgagtgtgtttaagtattg

SEQ ID NO: 32 - DNA, 21 bp, unidentified
  source 1..21: mol_type = other DNA
aaattacaaacatacttatca

SEQ ID NO: 33 - DNA, 41 bp, unidentified
  source 1..41: mol_type = other DNA
tctcctaataaaattccacctaagtgaaatggttatgtgtg

SEQ ID NO: 34 - DNA, 22 bp, unidentified
  source 1..22: mol_type = other DNA
tctcctaataaaattccaccta

SEQ ID NO: 35 - DNA, 46 bp, unidentified
  source 1..46: mol_type = other DNA
aatagtgtttttaatatttgttaagtacaattttattaccatcaat

SEQ ID NO: 36 - DNA, 19 bp, unidentified
  source 1..19: mol_type = other DNA
tttaacatataataaacca

SEQ ID NO: 37 - DNA, 18 bp, unidentified
  source 1..18: mol_type = other DNA
ggttaatgttaatgtatt

SEQ ID NO: 38 - DNA, 22 bp, unidentified
  source 1..22: mol_type = other DNA
ttttaattttagttatggtaga

SEQ ID NO: 39 - DNA, 18 bp, unidentified
  source 1..18: mol_type = other DNA
aaacaacaacaaacacaa

SEQ ID NO: 40 - DNA, 57 bp, unidentified
  source 1..57: mol_type = other DNA
ccatataaaaaataaaaaacctattactaaattccaatggtattattattgttgaag

SEQ ID NO: 41 - DNA, 45 bp, unidentified
  source 1..45: mol_type = other DNA
atgttaataggaataggtttttgtaacaaactaaaattactaacc

SEQ ID NO: 42 - DNA, 18 bp, unidentified
  source 1..18: mol_type = other DNA
caaaaaactttttaaact

SEQ ID NO: 43 - DNA, 26 bp, unidentified
  source 1..26: mol_type = other DNA
atataattaagttaatttttttttgg

SEQ ID NO: 44 - DNA, 17 bp, unidentified
  source 1..17: mol_type = other DNA
ggattattggtggaatt

SEQ ID NO: 45 - DNA, 18 bp, unidentified
  source 1..18: mol_type = other DNA
ttaatatcacaacatcct

SEQ ID NO: 46 - DNA, 48 bp, unidentified
  source 1..48: mol_type = other DNA
cacacaaacaatctaaaaaaaacaataaaaaatggtttgttttgtagg

SEQ ID NO: 47 - DNA, 46 bp, unidentified
  source 1..46: mol_type = other DNA
gttttatgtggttatttaatttagtatccacaaaaaatcacaactc

SEQ ID NO: 48 - DNA, 17 bp, unidentified
  source 1..17: mol_type = other DNA
aactaaaccacatcaaa

SEQ ID NO: 49 - DNA, 21 bp, unidentified
  source 1..21: mol_type = other DNA
ttgattagattgtttttagaa

SEQ ID NO: 50 - DNA, 21 bp, unidentified
  source 1..21: mol_type = other DNA
tgtataatttgtttttagtgt

SEQ ID NO: 51 - DNA, 21 bp, unidentified
  source 1..21: mol_type = other DNA
tttttatcctttttaaactct

SEQ ID NO: 52 - DNA, 46 bp, unidentified
  source 1..46: mol_type = other DNA
aatcaaccacattcccaaaaatattttagtgttttttggaatgttg

SEQ ID NO: 53 - DNA, 56 bp, unidentified
  source 1..56: mol_type = other DNA
tataggtgttattaaattggatgatattttatatacatcaatatacttattcaaca

SEQ ID NO: 54 - DNA, 16 bp, unidentified
  source 1..16: mol_type = other DNA
acttccataccaatac

SEQ ID NO: 55 - DNA, 22 bp, unidentified
  source 1..22: mol_type = other DNA
gatttaaattttaaagattaag

SEQ ID NO: 56 - DNA, 20 bp, unidentified
  source 1..20: mol_type = other DNA
gcasagagacttgaagatgt

SEQ ID NO: 57 - DNA, 20 bp, unidentified
  source 1..20: mol_type = other DNA
cagtttaactgctytgtcca

SEQ ID NO: 58 - DNA, 42 bp, unidentified
  source 1..42: mol_type = other DNA
tcagaggtgacaggattggtctagaataccgatcttgaggct

SEQ ID NO: 59 - DNA, 42 bp, unidentified
  source 1..42: mol_type = other DNA
gatttgtgttcacgctcaccgtccattcccattaagggcatt

SEQ ID NO: 60 - DNA, 20 bp, unidentified
  source 1..20: mol_type = other DNA
gtctttagccattccatgag

SEQ ID NO: 61 - DNA, 17 bp, unidentified
  source 1..17: mol_type = other DNA
ggactgcagcgtagacg

SEQ ID NO: 62 - DNA, 18 bp, unidentified
  source 1..18: mol_type = other DNA
aacacaagctttcggcag

SEQ ID NO: 63 - DNA, 22 bp, unidentified
  source 1..22: mol_type = other DNA
gaaatttggatctttgtcatcc

SEQ ID NO: 64 - DNA, 41 bp, unidentified
  source 1..41: mol_type = other DNA
tgcggccaatgtttgtaatcagccaaggaaattttggggac

SEQ ID NO: 65 - DNA, 38 bp, unidentified
  source 1..38: mol_type = other DNA
gcattggcatggaagtcactttgatggcacctgtgtag

SEQ ID NO: 66 - DNA, 19 bp, unidentified
  source 1..19: mol_type = other DNA
ttccttgtctgattagttc

SEQ ID NO: 67 - DNA, 19 bp, unidentified
  source 1..19: mol_type = other DNA
ttccttgtctgattagttc

SEQ ID NO: 68 - DNA, 21 bp, unidentified
  source 1..21: mol_type = other DNA
tccagatgaggatgaagaaga

SEQ ID NO: 69 - DNA, 21 bp, unidentified
  source 1..21: mol_type = other DNA
agtctgaacaactggtgtaag

SEQ ID NO: 70 - DNA, 41 bp, unidentified
  source 1..41: mol_type = other DNA
agagcagcagaagtggcacaggtgattgtgaagaagaagag

SEQ ID NO: 71 - DNA, 40 bp, unidentified
  source 1..40: mol_type = other DNA
tcaacctgaagaagagcaagaactgattgtcctcactgcc

SEQ ID NO: 72 - DNA, 22 bp, unidentified
  source 1..22: mol_type = other DNA
ctcatattgagttgatggctca

SEQ ID NO: 73 - DNA, 22 bp, unidentified
  source 1..22: mol_type = other DNA
acaaactgttggtcaacaagac

SEQ ID NO: 74 - DNA, 19 bp, unidentified
  source 1..19: mol_type = other DNA
tggctactaccgaagagct

SEQ ID NO: 75 - DNA, 20 bp, unidentified
  source 1..20: mol_type = other DNA
tgcagcattgttagcaggat

SEQ ID NO: 76 - DNA, 41 bp, unidentified; identical to SEQ ID NO: 15
  source 1..41: mol_type = other DNA

SEQ ID NO: 77 - DNA, 40 bp, unidentified; identical to SEQ ID NO: 16
  source 1..40: mol_type = other DNA

SEQ ID NO: 78 - DNA, 25 bp, unidentified
  source 1..25: mol_type = other DNA
ggactgagatctttcattttaccgt

SEQ ID NO: 79 - DNA, 21 bp, unidentified
  source 1..21: mol_type = other DNA
actgagggagccttgaataca

SEQ ID NO: 80 - DNA, 20 bp, unidentified
  source 1..20: mol_type = other DNA
ccactagaggagctactgta

SEQ ID NO: 81 - DNA, 18 bp, unidentified
  source 1..18: mol_type = other DNA
tgacaagctacaacacgt

SEQ ID NO: 82 - DNA, 48 bp, unidentified
  source 1..48: mol_type = other DNA
aggtgagggttttctacatcactatattggaacaagcaaattctatgg

SEQ ID NO: 83 - DNA, 43 bp, unidentified
  source 1..43: mol_type = other DNA
atgggttgggattatcctaaatgtgtgcgagcaagaacaagtg

SEQ ID NO: 84 - DNA, 25 bp, unidentified
  source 1..25: mol_type = other DNA
cagtttttaacatgttgtgccaacc

SEQ ID NO: 85 - DNA, 21 bp, unidentified
  source 1..21: mol_type = other DNA
tagagccatgcctaacatgct

SEQ ID NO: 86 - DNA, 18 bp, unidentified
  source 1..18: mol_type = other DNA
tgcttcagtcagctgatg

SEQ ID NO: 87 - DNA, 22 bp, unidentified
  source 1..22: mol_type = other DNA
ttaaattgtcatcttcgtcctt

SEQ ID NO: 88 - DNA, 41 bp, unidentified
  source 1..41: mol_type = other DNA
tcagtactagtgcctgtgcccacaatcgtttttaaacgggt

SEQ ID NO: 89 - DNA, 43 bp, unidentified
  source 1..43: mol_type = other DNA
tcgtatacagggcttttgacatctatcttggaagcgacaacaa

SEQ ID NO: 90 - DNA, 17 bp, unidentified
  source 1..17: mol_type = other DNA
ctgcacttacaccgcaa

SEQ ID NO: 91 - DNA, 22 bp, unidentified
  source 1..22: mol_type = other DNA
gtagctggttttgctaaattcc

SEQ ID NO: 92 - DNA, 21 bp, unidentified
  source 1..21: mol_type = other DNA
tccagatgaggatgaagaaga

SEQ ID NO: 93 - DNA, 21 bp, unidentified
  source 1..21: mol_type = other DNA
agtctgaacaactggtgtaag

SEQ ID NO: 94 - DNA, 41 bp, unidentified; identical to SEQ ID NO: 70
  source 1..41: mol_type = other DNA

SEQ ID NO: 95 - DNA, 40 bp, unidentified; identical to SEQ ID NO: 71
  source 1..40: mol_type = other DNA

SEQ ID NO: 96 - DNA, 22 bp, unidentified
  source 1..22: mol_type = other DNA
ctcatattgagttgatggctca

SEQ ID NO: 97 - DNA, 22 bp, unidentified
  source 1..22: mol_type = other DNA
acaaactgttggtcaacaagac

SEQ ID NO: 98 - DNA, 18 bp, unidentified
  source 1..18: mol_type = other DNA
cggtggacaaattgtcac

SEQ ID NO: 99 - DNA, 51 bp, unidentified
  source 1..51: mol_type = other DNA
aattctaatacgactcactatagggcccataatcggtggacaaattgtcac

SEQ ID NO: 100 - DNA, 22 bp, unidentified
  source 1..22: mol_type = other DNA
cttctctggatttaacacactt

SEQ ID NO: 101 - DNA, 51 bp, unidentified
  source 1..51: mol_type = other DNA
tcagcacacaaagccaaaaatttatttttctgtgcaaaggaaattaaggag

SEQ ID NO: 102 - DNA, 49 bp, unidentified
  source 1..49: mol_type = other DNA
tattggtggagctaaacttaaagccttttctgtacaatccctttgagtg

SEQ ID NO: 103 - DNA, 28 bp, unidentified
  source 1..28: mol_type = other DNA
ttacaagcttaaagaatgtctgaacact

SEQ ID NO: 104 - DNA, 27 bp, unidentified
  source 1..27: mol_type = other DNA
ttgaatttaggtgaaacatttgtcacg

SEQ ID NO: 105 - DNA, 19 bp, unidentified
  source 1..19: mol_type = other DNA
gcggtggatgatgtggatt

SEQ ID NO: 106 - DNA, 52 bp, unidentified
  source 1..52: mol_type = other DNA
aattctaatacgactcactatagggcccataatgcggtggatgatgtggatt

SEQ ID NO: 107 - DNA, 20 bp, unidentified
  source 1..20: mol_type = other DNA
ccggcagtctcattagagtg

SEQ ID NO: 108 - DNA, 53 bp, unidentified
  source 1..53: mol_type = other DNA
aattctaatacgactcactatagggcccataatccggcagtctcattagagtg

SEQ ID NO: 109 - DNA, 41 bp, unidentified
  source 1..41: mol_type = other DNA
ctcctccgtctccggaggattcgatgcaacgcgaagaacct

SEQ ID NO: 110 - DNA, 40 bp, unidentified
  source 1..40: mol_type = other DNA
tcgtcagctcgtgtcgtgagatcccaaccgaatgatggca

SEQ ID NO: 111 - DNA, 21 bp, unidentified
  source 1..21: mol_type = other DNA
cgcacatgtcaaaaccaggta

SEQ ID NO: 112 - DNA, 19 bp, unidentified
  source 1..19: mol_type = other DNA
gttgggttaagtcccgcaa